(19) United States
(12) Patent Application Publication
(10) Pub. No.: US 2014/0017637 A1
(43) Pub. Date: Jan. 16, 2014

(54) DENTAL ADHESIVE COMPRISING A COATED POLYMERIC COMPONENT

(71) Applicants: David K. Cinader, Jr., Walnut, CA (US); Bill H. Dodge, Finlayson, MN (US); Bradley W. Eaton, Woodbury, MN (US)

(72) Inventors: David K. Cinader, Jr., Walnut, CA (US); Bill H. Dodge, Finlayson, MN (US); Bradley W. Eaton, Woodbury, MN (US)

(73) Assignee: 3M IINNOVATIVE PROPERTIES COMPANY, St. Paul, MN (US)

(21) Appl. No.: 14/007,033

(22) PCT Filed: Mar. 19, 2012
(86) PCT No.: PCT/US12/29595

§ 371 Date: Sep. 24, 2013

Related U.S. Application Data
(60) Provisional application No. 61/466,958, filed on Mar. 24, 2011.

(51) Int. Cl. A61K 6/00; A61C 13/271; A61K 6/087; A61C 7/28; A61K 6/06; A61C 5/08; A61C 5/04

(52) U.S. Cl. CPC A61K 6/0023; A61C 5/08; A61C 13/26; A61C 5/04; A61C 7/282; A61K 6/0631; A61K 6/0002; A61K 6/087
USPC 433/228.1; 433/226; 427/2.29

(57) ABSTRACT

Dental materials are provided in which a conformal coating is disposed on the inner surfaces of a dental composite. The conformal coating can be subsequently surface modified to provide a chemical bond between the compressible component and a hardenable resin, resulting in increased bond strength and reduced bond strength variability. Use of a thin conformal layer can provide a surprising enhancement in bond reliability while minimizing the collateral effect on the handling properties of the unhardened composite. The conformal coating can also improve the compressibility and decrease rebound in the overall dental composite during a bonding procedure and reduce the amount of extractable components.

### 1. FIELD OF THE INVENTION

Composite materials and related methods useful in the field of dentistry are provided. More particularly, dental composites, assemblies, kits and related methods are provided for intraoral bonding applications.

### 2. DESCRIPTION OF THE RELATED ART

Dental composites are engineered materials made from two or more chemically dissimilar constituent materials. Typically, these materials include a synthetic resin component along with one or more filler components.

In dentistry, these composites are commonly used as restorative materials as well as adhesives. The resin component is usually a liquid at ambient temperatures and can be hardened on demand at an appropriate time by the user. The filler component, on the other hand, is generally a solid at ambient temperatures and provides strength, abrasion resistance, and structure to the composite after the resin component is hardened. Common fillers include silicon dioxide (silica) and quartz, though various other glasses and glass ceramics may be used. The filler component can also be used to control the texture and handling properties of the composite, with filler loading generally ranging from a few percent to 70 percent or more by weight.

In a broad sense, dental composites are bonded either directly or indirectly to a patient's dental structure. When dental composites are used in bonding a dental article to a patient's dental structure, bond strength should be adequately high to maintain the integrity of the dental article during its expected lifetime. It is ideal for most dental restoratives to last as long as possible. In an orthodontic context, these dental composites are commonly referred to as orthodontic adhesives and are used to bond orthodontic appliances, such as bands or brackets, to teeth.

### 3. SUMMARY OF THE INVENTION

The adhesive and cohesive strength of a dental composite are affected to a significant degree by the surface chemistry of the materials selected. In particular, the chemical compatibility or lack thereof between the constituents of the composite can bear significantly on adhesive and cohesive strength. Since a composite material is heterogeneous by definition, cohesive and adhesive strength can be benefitted by selecting components that display a high degree of interfacial bond strength.

This basis for materials selection, however, can be constrained or even frustrated by bulk property requirements for each component. For example, U.S. Patent Application Publication No. 2009/0233252 (Cinader) describes dental assemblies that combine compressible materials and hardenable components to achieve properties approaching those of traditional filler-based composites. Pairing a particular compressible material with a particular resin could provide ideal stiffness and handling properties, and yet provide less-than-ideal interfacial bond strength, or vice versa. Thus, there is a compelling advantage in achieving high interfacial bond strength while preserving the desirable bulk properties of each component.

This dilemma of being forced to choose composite components based either on their bulk properties or surface properties can be resolved by depositing a thin conformal coating to the compressible material. This conformal coating can be subsequently surface modified to provide a chemical bond between the compressible component and the hardenable component, resulting in increased bond strength and reduced bond strength variability. As a further advantage, the conformal coating provides barrier properties despite being extremely thin and having minimal impact on the handling properties of the unhardened composite. The presence of the coating was also found to improve compressibility, reduce rebound, and mitigate the levels of extractable components after hardening, with respect to the same characteristics in uncoated composites.

In one aspect, a dental composite is provided. The dental composite comprises a compressible material; and a conformal coating disposed on at least a portion of the compressible material.

In another aspect, a dental assembly is provided, comprising: a dental article having an outer surface for attachment to a tooth; and an adhesive in contact with the outer surface, the adhesive comprising: a compressible material; and a conformal coating disposed on at least a portion of the compressible material.

In still another aspect, a dental assembly is provided, comprising: a dental article having an outer surface for attachment to a tooth; and an adhesive at least partially coated on the outer surface, the adhesive comprising: a polymeric component; and a conformal coating disposed on at least a portion of the polymeric component.

In yet another aspect, a method of making a dental composite is provided, comprising: applying a conformal coating to at least a portion of a compressible material to enhance the wetting properties of the compressible material; and placing a hardenable composition in contact with the inorganic coating.

In yet another aspect, a method of enhancing the bond strength of a dental composite containing a polymeric component is provided, comprising: applying a conformal coating to a portion of the polymeric component; and placing a hardenable composition in contact with the conformal coating.

### 4. BRIEF DESCRIPTION OF THE DRAWINGS

FIG. 1A is an electron micrograph showing a compressible material used in a dental assembly according to one embodiment.

FIG. 1B is a cross-sectional view of a single fiber strand of the compressible material shown in FIG. 1A.

FIG. 1C is a side view of another embodiment showing a dental article having a compressible material attached thereon.

FIG. 2 is a perspective view of a dental assembly according to still another embodiment;

FIG. 3 is an occlusal view of the dental assembly in FIG. 2;

FIG. 4 is a perspective view of a packaged article including the orthodontic assembly of FIGS. 2-3 located in a container in which the cover has been partially opened.

FIG. 5 is an occlusal view showing a step in bonding an orthodontic appliance to a tooth structure, and illustrates an orthodontic appliance, a compressible material, and a tooth structure prior to bonding the orthodontic appliance to the tooth structure.

FIG. 6 is an occlusal view showing another step in bonding an orthodontic appliance to a tooth structure, and illustrates an orthodontic appliance, a compressible material, and a tooth structure during or subsequent to bonding the orthodontic appliance to the tooth structure.

FIG. 7 is a side cross-sectional view showing the act of applying a dental appliance to a tooth structure using a placement device used with an indirect bonding method.

### 5. DETAILED DESCRIPTION OF THE ILLUSTRATIVE EMBODIMENTS

Various embodiments are described herein by way of illustration and example. These embodiments include dental composites with coated surfaces, assemblies, kits and methods that are broadly related to the field of dentistry, and include particular applications in the area of orthodontics. Optionally, these dental composites include at least one hardenable dental composition (or resin). The hardenable dental composition, in turn, may include a hardenable component, such as an ethylenically unsaturated compound (acidic or non-acidic), epoxy or vinyl ether compound, or glass ionomer. Additionally, the dental composition may include a hardener, such as a photoinitiator or redox initiator system that can be activated to harden the hardenable composition. Finally, the overall dental composition may include fillers, photobleachable or thermochromic dyes, and/or other optional miscellaneous additives. Each of the above is examined in greater detail under the headings and subheadings that follow.

Dental Composites with Coated Surfaces

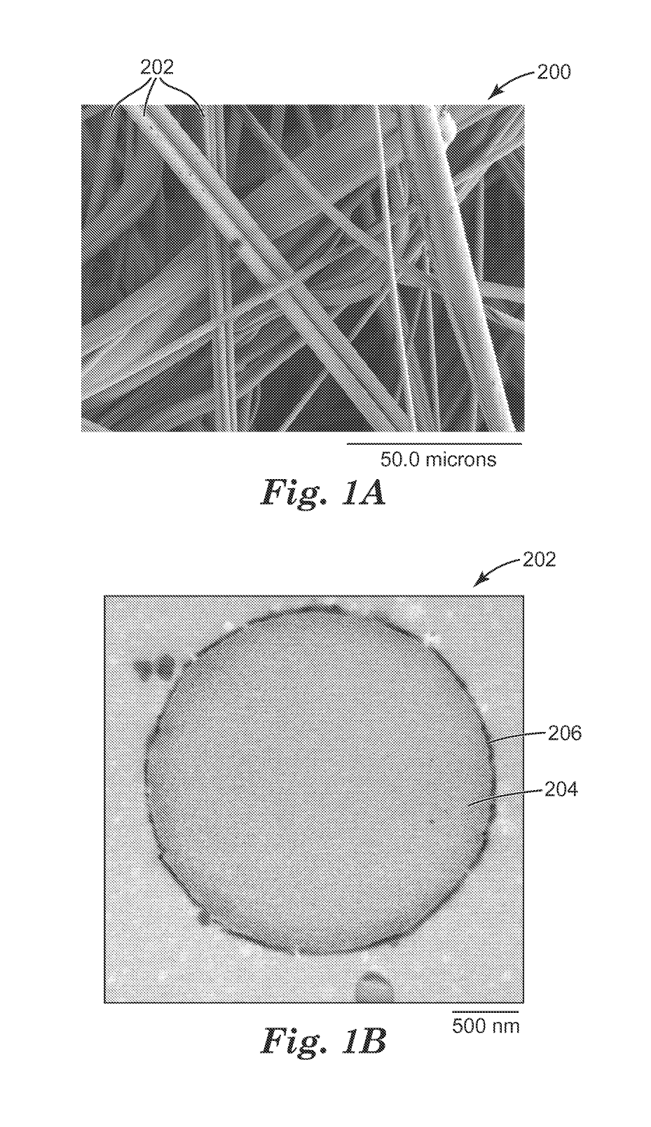

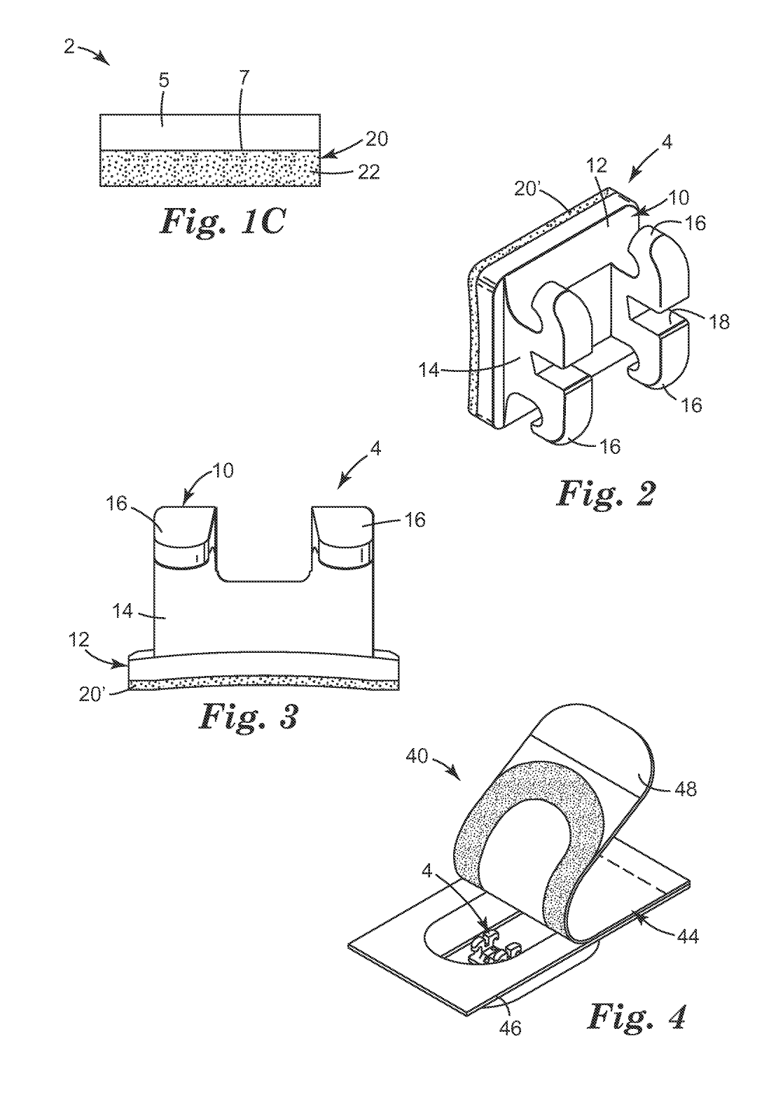

An exemplary compressible material useful in a dental composite is shown in FIGS. 1A and 1B and broadly designated by the numeral 200. In the particular embodiment shown, the compressible material 200 is a nonwoven material including a multiplicity of polymeric fibers 204. A conformal coating 206 is disposed on the compressible material 200, thereby providing a multiplicity of coated fibers 202. FIG. 1B shows a cross-sectional micrograph of a single strand of the coated fibers 202. As shown, the conformal coating 206 extends along the entire circumference of an uncoated fiber 204, providing a substantially uniform barrier layer separating the uncoated fiber 204 from its surrounding environment.

As used herein, a “conformal coating” refers to a relatively thin coating of material that adheres well to and conforms closely to the shape of an underlying substrate. In preferred embodiments, the conformal coating is disposed on essentially all surfaces of the substrate, including the inner surfaces of the compressible material 200 that are hidden from view.

As used herein, a “compressible material” broadly refers to a material that is significantly reduced in volume upon application of pressures typically employed to place and/or position a dental article on a tooth structure. Forces typically employed to place and/or position a dental article on a tooth structure generally range from 0.5 to 5 pound-force, as applied to a bonding base of area 0.106 square centimeters (0.0164 square inches). This corresponds to calculated pressures ranging from 0.2 to 2.0 megapascals. The ratio of the compressed volume/initial volume (i.e., compressibility) will vary depending on the compressible material used. In some embodiments, the compressibility is typically at most 0.9, at most 0.7, or at most 0.5. In some embodiments, the compressibility is at least 0.001, at least 0.01, or at least 0.1.

In a preferred embodiment, the compressible material 200 is a nonwoven material made using a standard meltblown fiber forming process. Such a process is described in U.S. Patent Application Publication No. 2006/0096911 (Brey et al.) Blown microfibers are generally created by a molten polymer that enters and flows through a die, the flow being distributed across the width of the die in the die cavity. The polymer exits the die through a series of orifices as filaments. In one embodiment, a heated air stream passes through air manifolds and an air knife assembly adjacent to the series of polymer orifices that form the die exit. This heated air stream is adjusted for both temperature and velocity to attenuate the polymer filaments down to the desired fiber diameter. The fibers can then be conveyed in this turbulent air stream towards a rotating surface where they were collected to form a web.

Alternatively, the nonwoven material may be made using any of a number of other manufacturing methods known in the art. For example, the fibers may be electrospun or spunbond. As a further alternative, the fibers could be drawn down to form staple fiber webs, crimped, and then cut into shorter lengths to be processed into a nonwoven web.

Nonwoven materials can be particularly suitable as compressible materials for dental composites because they are highly open structures that allow a resin to permeate throughout the bulk of the nonwoven material. Nonwovens can also be manufactured with a wide range of effective fiber diameters (EFD), as determined by the method set forth in Davies, C. N., “The Separation of Airborne Dust and Particles,” Institution of Mechanical Engineers, London Proceedings 1B, 1952. Advantageously, EFD can be used adjust the density, texture and handling properties of the composite. In some embodiments, the nonwoven has an average EFD of at least 0.1 micrometers, at least 0.5 micrometers, at least 1.0 micrometers, at least 2 micrometers, or at least 2.5 micrometers. In some embodiments, the nonwoven has an average EFD that is at most 20 micrometers, at most 15 micrometers, at most 10 micrometers, at most 8 micrometers, or at most 6 micrometers.

A nonwoven mat or fabric can be made from any of a variety of polymeric materials, including thermoplastic polyurethanes, polybutylenes, polyesters, polyolefins (e.g. polyethylene and polypropylene), polyesters, styrenic copolymers, nylon, and combinations thereof. In dental composite applications, polypropylene was found to be especially advantageous because it resisted absorption of most hardenable dental compositions and provided a relatively high degree of compressibility.

While a nonwoven material is shown in FIG. 1A, the compressible material 200 can also be made from other porous materials. For example, the compressible material 200 can be provided by foams (for example, cellulose foams, glass foams, polymeric foams, and combinations thereof), sponges, glass fibers (e.g., glass wool), ceramic fibers, cotton fibers, cellulose fibers, woven mats, scrims, and combinations thereof.

Various methods are capable of applying the conformal coating to the compressible material 200. One particularly preferred method is stepwise atomic layer deposition (ALD), as described, for example, in International Patent Publication Nos. WO2011/037831 (Dodge) and WO2011/037798 (Dodge). ALD provides advantages compared with other technologies. First, this method uses a series of sequential self-limiting surface chemical reactions to build up the coating, thereby allowing for precise control over the final thickness. Second, this method employs a reactive gas that is capable of permeating and coating porous materials and constructions. For example, two or more reactive gases can be iteratively transmitted through the compressible material to induce two or more self-limiting reactions on the surface of the compressible material. Nanoscale coatings having superior conformability and substantially uniform thickness are possible, since the deposition is non-directional and does not require a line of sight between the deposition apparatus and substrate. Finally, ALD can be used to deposit coatings of a variety of chemically diverse materials.

In one preferred embodiment, ALD is used to deposit a conformal aluminum oxide (Al₂O₃) coating using the binary reaction 2Al(CH₃)₃+3H₂O→Al₂O₃+6CH₄. This can be split into the following two surface half-reactions:

AlOH*+Al(CH₃)₃→AlOAl(CH₃)₂*+CH₄ (1)

AlCH₃*+H₂O→AlOH*+CH₄ (2)

In reactions (1) and (2) above, the asterisks denote surface species. In reaction (1), Al(CH₃)₃ reacts with the hydroxyl (OH*) species, depositing aluminum and methylating the surface. Reaction (1) stops after essentially all the hydroxyl species have reacted with Al(CH₃)₃. Then, in reaction (2), H₂O reacts with the AlCH₃* species and deposits oxygen and rehydroxylates the surface. Reaction (2) stops after essentially all the methyl species have reacted with H₂O. Because each reaction is self-limiting, deposition occurs with atomic layer control.

In some embodiments, the conformal coating has a thickness of at least 0.5 nanometers, at least 1 nanometer, at least 2 nanometers, at least 3 nanometers, or at least 4 nanometers. In some embodiments, the conformal coating has a thickness of at most 50 nanometers, at most 20 nanometers, at most 15 nanometers, at most 10 nanometers, or at most 8 nanometers. Coating growth in ALD can be monitored and recorded using any known method, including use of a quartz crystal microbalance.

Materials capable of being coated using ALD include binary materials, i.e., materials of the form QₓR_{y}, where Q and R represent different atoms and x and y are selected to provide an electrostatically neutral material. Suitable binary materials include inorganic oxides (such as silicon dioxide and metal oxides such as zirconia, alumina, silica, boron oxide, yttria, zinc oxide, magnesium oxide, titanium dioxide and the like), inorganic nitrides (such as silicon nitride, AlN and BN), inorganic sulfides (such as gallium sulfide, tungsten sulfide and molybdenum sulfide), as well as inorganic phosphides. In addition, various metal coatings are also possible, including cobalt, palladium, platinum, zinc, rhenium, molybdenum, antimony, selenium, thallium, chromium, platinum, ruthenium, iridium, germanium tungsten, and combinations and alloys thereof.

ALD may also be used to coat filler particles present in a dental composite. In some embodiments, an inorganic coating is deposited on a polymeric filler such as a polymethylmethacrylate filler. Monodisperse polymeric fillers can be made, for example, using an emulsion or suspension polymerization. These softer fillers can not only display bond strength on par with silica-based fillers but also present some significant advantages in remnant adhesive cleanup, as described in International Patent Publication No. WO2009/045752 (Kalgutkar, et al.). Advantageously, ALD can be used to apply a substantially uniform coatings to particles by performing the deposition process in, for example, a fluidized bed configuration.

Self-limiting surface reactions can also be used to grow organic polymer films or coatings. This type of growth is often described as molecular layer deposition (MLD), since a molecular fragment is deposited during each reaction cycle. MLD methods have been developed for the growth of polymers such as polyamides, which uses dicarboxylic acid and diamines as reactants. Known approaches to MLD, involving heterobifunctional and ring-opening precursors, can also be used. Further details concerning MLD are described in George, et al., Accounts of Chemical Research (2009) 42, 498 (2009).

Other deposition methods can also be used to deposit a coating onto a compressible material. For example, layer-by-layer polyelectrolyte coating can be used to prepare a conformal coating with precisely controlled thickness. This method involves depositing alternating cationic and anionic polyelectrolyte layers from aqueous solution to incrementally build up a surface coating. Additional details concerning layer-by-layer polyelectrolyte coating are provided in U.S. Patent Application Publication No. 2010/080841 (Porbeni, et al.).

As suggested by the methods described above, the conformal coating 206 can be either polymeric (organic) or ceramic (inorganic). In the context of dental composites, applying an inorganic coating to the non woven mat was found to provide one or more surprising technical advantages.

First, the presence of the inorganic material at the surface of the polymeric fibers substantially changes the surface chemistry of the fibers. The conformal inorganic coating can be present in an amount sufficient to enhance the wetting behavior of the compressible material. For thin layer depositions, the extent of the modification can also be tailored by controlling layer thickness. Moreover, atomic layer deposition is a quantitative deposition method, thus providing precise control over layer thickness that is superior to conventional methods such as physical vapor deposition or sputtering. Having a tailored wetting behavior can help ensure that the fibers are substantially uniformly coated by the hardenable component (or resin). Enhanced wettability can also facilitate uptake and/or saturation of the resin in the nonwoven material. All of these factors can positively affect bond strength and bonding predictability of the adhesive assembly.

Second, the inorganic coating can provide a chemistry for further surface modification, such as silane treatment (or silanation). Advantageously, a silanated surface can allow for chemical bonding between the compressible material and the resin. Unlike previously known adhesive assemblies, these assemblies allow for both mechanical and chemical bonding between the compressible material and the resin. This can significantly enhance bond strength and bond reliability. A unique feature of the provided dental composites is that covalent bonding occurs not only at the interface between the resin and the inorganic coating but also the interface between the inorganic coating and the fibers of nonwoven material. Further options and advantages of silane treatment are described in U.S. Provisional Patent Application Ser. No. 61/383,353 (Tzou, et al.), entitled, “Functionalized Adhesive Coated Orthodontic Appliances” and filed on Sep. 16, 2010.

Third, the inorganic coating serves as a chemical barrier between the resin and the fibers of the nonwoven material. This is especially significant here, where the nonwoven mat is polymeric and has the potential to contain oligomers, additives, stabilizers, or other small molecules capable of leaching out of the polymer. By substantially uniformly coating the fibers, the inorganic coating can minimize or prevent these extractable components from leaving the fibers in the presence of the resin or a solvent. Further, the coating can also serve as a barrier to certain gases, such as oxygen, which could diffuse out of nonwoven fibers and inhibit resin polymerization. Solvent extraction studies, which showed that adhesive assemblies with coated polypropylene fibers displayed reduced loss of mass compared with adhesives assemblies with uncoated polypropylene fibers (see Examples).

Fourth, coating the compressible material with a thin conformal layer can also reduce the level of rebound in the overall composite. This can be especially beneficial with respect to orthodontic adhesive applications, where the rebound should generally be minimized. Low rebound is desirable not only to express as accurately as possible the in-out prescription of the appliance but also to alleviate the risk of voids or cavitation in the composite result from air entering in the composite upon rebound. Surprisingly, nonwoven materials coated with a conformal alumina coating ranging in thickness from 4 to 8 nanometers were observed to display not only excellent wettability and barrier properties but also decreased rebound compared with equivalent uncoated materials.

Finally, application of an inorganic coating on the nonwoven materials provides a convenient handle to modify the bulk mechanical properties of the adhesive assembly. For example, the ALD coating can be used to stiffen the fibers of the nonwoven thereby stiffening the compressible material. Alternatively, these coatings can be used to precisely alter the permeability of the compressible material, or the level of rebound which occurs when the material is compressed and then allowed to relax. Each of these represents a significant bulk property that can be adjusted to provide optimal adhesive handling

Various embodiments display one or more of the above described advantages.

### Assemblies and Kits

An exemplary dental assembly is shown in FIG. 1C. In FIG. 1C, a dental assembly 2 includes a dental article 5 having an outer surface 7 for bonding to a tooth structure. The exemplary article 5 can represent a wide variety of dental articles including, but not limited to, crowns, bridges, veneers, inlays, onlays, fillings, orthodontic appliances (e.g. brackets, bands, molar tubes, sheaths, cleats, and buttons) and devices, and prostheses (e.g., partial or full dentures). The exemplary assembly 2 is in contact with a dental composite 20. The dental composite 20 includes a compressible material 22 in contact with the surface 7 of the dental article 5. Although not visible in FIG. 1C, the dental composite 20 includes a hardenable dental composition absorbed in the compressible material 22 to provide an orthodontic adhesive.

Optionally, the compressible material 22 is saturated with the hardenable dental composition, although this need not be the case. In some embodiments, the compressible material 22 represents at least 2%, at least 4%, at least 6%, at least 8%, or at least 10% the total weight of the dental composite 20. In some embodiments, the compressible material 22 represents at most 40%, at most 30%, at most 25%, at most 20%, at most 15%, or at most 12% the total weight of the dental composite 20.

As shown, the compressible material 22 extends across the entire surface 7 of the dental article 5. The compressible material 22, in combination with the hardenable dental composition, can serve in whole or at least in part to securely fix the article 2 to a tooth structure by a bond having sufficient strength to resist unintended detachment from the tooth structure. The hardenable dental composition can be placed in contact with all or a portion of the compressible material 22 by methods known in the art including, but not limited to, coating, spraying, dipping, brushing, and the like.

In preferred embodiments, the compressible material 22 is essentially saturated with the hardenable dental composition. However, the hardenable dental composition can optionally be applied to compressible material 22 non-uniformly (e.g., applied to only one side of the compressible material). The hardenable dental composition can be patterned on compressible material 22. For example, an unfilled or lightly filled hardenable dental composition can be applied proximate the periphery of compressible material 22, and a filled hardenable dental composition can be applied proximate the center of compressible material 22.

Optionally, one part of a two-part hardenable dental composition (e.g., a chemical cure primer) can be applied to all or a portion of compressible material 22, and the second part of the two-part hardenable dental composition can be applied to a tooth surface. In other embodiments, one part of a redox pair can be coated on, adsorbed by, and/or embedded in the compressible material 22, and the other part of the redox pair can be included in the hardenable dental composition, which can be applied just prior to placement on the tooth.

In some embodiments, the dental composite 20 is attached by the manufacturer to surface 7 of dental article 5. The dental composite 20 can be attached to surface 7 of dental article 5 using an unhardened dental composition, a partially hardened dental composition, or a hardened dental composition. In preferred embodiments, compressible material 22 is mechanically bonded to surface 7 of dental article 5, chemically bonded to surface 7 of dental article 5, or a combination thereof.

As used herein, “mechanically bonded” means bonded or attached through physical means (e.g., using hooks, loops, protrusions, van der Waals interactions, ionic bonds, and the like, including combinations thereof), and in certain embodiments utilizing the undercuts provided by a wire mesh (e.g. on metal brackets) or glass grit (e.g., on ceramic brackets). As used herein, “chemically bonded” means bonded or attached through chemical means (e.g., via shared electron pairs such as covalent bonding, coordinate covalent bonding, acid-base interactions such as Brønsted-Lowry reactions, and the like, including combinations thereof). For example, a hardenable dental composition (e.g., a hardenable resin, glass ionomer, resin-modified glass ionomer, and/or epoxy) can be hardened to chemically bond the compressible material 22 to surface 7 of dental article 5. In certain embodiments, compressible material 22 can be surface treated (e.g., with a silane coupling agent) at a level sufficient to enhance the bond to surface 7 of dental article 5. In some embodiments, the compressible material 22 can be bonded to surface 7 of dental article 5 by melting or softening the compressible material.

In a preferred embodiment, the dental composite 20 is both mechanically and chemically bonded to the dental article 7 using a local “spot-binding” method described in U.S. Provisional Patent Application Ser. No. 61/428,498 (Cinader, et al.), entitled, “Bondable Dental Assemblies and Methods Including a Compressible Material” and filed on Dec. 30, 2010.

Attachment of dental composite 20 to the surface 7 of the dental article 5 can be enhanced by a sandblasting treatment as described, for example, in Akin-Nergiz et al., Fortschritte der Kieferorthopadie (1995) 56(1):49-55; Atsu et al., Angle Orthodontist (2006) 76(5):857-862; Mujagic et al., J. of Clinical Orthodontics (2005) 39(6):375-382; Newman et al., American J. of Orthodontics and Dentofacial Orthopedics (1995) 108(3):237-241; and Wiechmann, J. of Orofacial Orthopedics (2000) 61(4):280-291.

In brief, the treatment includes sandblasting the surface 7 with a silica-coated alumina sandblasting medium available as Rocatec Plus (3M Company, St. Paul, Minn.). The sandblasting treatment can be carried out using, for example, a blasting module available as Rocatec Jr. (3M Company, St. Paul, Minn.), with the module set at 2.8 bar for 2 to 3 seconds at a distance of one centimeter. A solution of silane (e.g., a silane in ethanol available as 3M ESPE Sil (3M Company, St. Paul, Minn.)) can then be applied to the treated surface 7 and allowed to dry at room temperature for at least 5 minutes. It is believed that the silane can further enhance the bonding of methacrylate-containing resins to the treated surface 7.

In some embodiments, the compressible material 22 is supplied having a hardenable dental composition therein and is supplied to the practitioner as a packaged article. In another embodiment, a practitioner can manually place a hardenable dental composition in contact with the conformal coating that is disposed on the compressible material 22. For example, the practitioner can apply a hardenable dental composition to the compressible material 22, or can dip or immerse the compressible material 22 in a hardenable dental composition.

The assembly 2 can optionally include additional layer(s) of dental compositions (e.g., orthodontic adhesives, orthodontic primers, or combinations thereof, which are not illustrated in FIG. 1C) in contact with compressible material 22. Specifically, such additional layer(s) can be between the surface 7 and the compressible material 22, on the compressible material 22 opposite the surface 7, or both. Such layers may or may not cover the same area, and may independently be discontinuous (e.g., a patterned layer) or continuous (e.g., non-patterned) materials extending across all or a portion of the compressible material 22.

FIGS. 2 and 3 show an exemplary dental assembly 4 that includes an orthodontic appliance 10 having a dental composite 20′ attached to the base thereof. In these figures, the orthodontic appliance 10 is a bracket, although other appliances such as buccal tubes, buttons, sheaths, bite openers, lingual retainers, bands, cleats, and other attachments may be similarly represented. The appliance 10 includes a base 12 and a body 14 extending outwardly from the base 12. The base 12 conforms to the tooth and can be made of metal, plastic, glass, ceramic, or combinations thereof. Optionally, the bonding surface of the base 12 includes a mesh-like structure or attached particles (such as shards, grit, spheres, or other structure that optionally includes undercuts) to provide mechanical retention. Alternatively, the base 12 is a custom resin base formed from one or more at least partially hardened dental composition layer(s). A pair of occlusal and gingival tiewings 16 are connected to body 14, and an elongated archwire slot 18 extends in a generally mesial-distal direction between the occlusal and gingival tiewings 16.

The base 12, body 14, and tiewings 16 may be made from any of a number of materials suitable for use in the oral cavity and having sufficient strength to withstand the correction forces applied during treatment. Suitable materials include, for example, metallic materials (such as stainless steel), ceramic materials (such as monocrystalline or polycrystalline alumina), and plastic materials (such as fiber-reinforced polycarbonate). Optionally, base 12, body 14, and tiewings 16 are integrally made as a unitary component.

The assemblies 2, 4 may be provided in a package or container that includes the dental article or appliance, as described herein. Exemplary containers known in the art are disclosed, for example, in U.S. Pat. Nos. 5,172,809 (Jacobs et al.) and 6,089,861 (Kelly et al.). In certain embodiments, the package can be an inverted blister with a foam inside that contacts the tie wings such that the appliance would be held in place and not slide on the liner. For example, rather than contacting the “bottom” of the blister well, the appliance can be positioned in the package such that it rests on the lid, and foam can be placed in the bottom of the blister such that it contacts the tie wings and holds the bracket in place. A conformal foam can also be placed on the lid of the blister, as described in PCT Publication No. WO2011/153039.

The external surface of the dental composite 20′ optionally has a concave configuration, and optionally has a compound concave configuration matching the convex configuration of the outer surface of the tooth intended for use with the appliance 10. As one example, the dental composite 20′ may have a substantially uniform thickness and the outer surface of the base 12 may have a concave configuration, such that the external surface of the compressible material when attached to the outer surface of the base 12 has a concave configuration that generally matches of the concave configuration of the outer surface of the base 12. As another example, the outer surface of the base 12 may have a generally planar configuration that matches a generally planar configuration of a facing surface of the compressible material 22, while the external surface of the compressible material 22 may have a concave configuration that inversely matches the convex configuration of the tooth surface. Other constructions are also possible, including, for example, constructions in which the thickness of the dental composite 20′ varies corresponding to different positions along the base 12.

As previously mentioned with respect to the assembly 2, the assembly 4 optionally includes additional layer(s) of dental compositions (e.g., orthodontic adhesives, orthodontic primers, or combinations thereof, not illustrated in FIGS. 2 and 3) in contact with the dental composite 20′.

Referring now to FIG. 4, an exemplary embodiment of a packaged assembly 40 is shown including the assembly 4, which includes an orthodontic appliance and dental composite similar to the exemplary embodiment illustrated in FIGS. 2 and 3. The packaged assembly 40 includes a package 44, which in turn includes a container 46 and cover 48. The cover 48 is releasably connected to the container 46 as initially provided, and is peeled from the container 46 to open the package for removal of the assembly 4. In FIG. 4, the cover 48 has been peeled back from container 46 to partially open package 44.

The package can provide excellent protection against degradation of optional hardenable dental composition(s) (e.g., photocurable materials), even after extended periods of time. Such containers are particularly useful for embodiments in which the optional hardenable dental composition optionally includes dyes that impart a color changing feature to the adhesive. Such containers preferably effectively block the passage of actinic radiation over a broad spectral range, and as a result, the optional dental compositions do not prematurely lose color during storage.

In preferred embodiments, the container 46 comprises a polymer and metallic particles. As an example, the container 46 may be made of polypropylene that is compounded with aluminum filler or receives an aluminum powder coating as disclosed, for example, in U.S. Patent Application Publication No. 2003/0196914 (Tzou et al.). The combination of polymer and metallic particles provides a highly effective barrier to the passage of actinic radiation. Such containers also exhibit good vapor barrier properties. As a result, the color-sensitive dyes are less likely to fade and rheological characteristics of the hardenable dental composition(s) are less likely to change over extended periods of time. For example, the improved vapor barrier properties of such containers provide substantial protection against degradation of the handling characteristics of adhesives so that the dental compositions do not prematurely cure or dry or become otherwise unsatisfactory. Suitable covers 48 for such containers can be made of any material that is substantially opaque to the transmission of actinic radiation so that the dental compositions do not prematurely cure. Examples of suitable materials for the cover 48 include laminates of aluminum foil and polymers. For example, the laminate may comprise a layer of polyethyleneterephthalate, adhesive, aluminum foil, adhesive and oriented polypropylene.

In some embodiments, a packaged assembly including an orthodontic appliance, a coated dental composite, and a hardenable dental composition may further include a release substrate as described, for example, in U.S. Pat. No. 6,183,249 (Brennan et al.).

In some embodiments, a package can include a set of assemblies including orthodontic appliances, where at least one of the assemblies includes an appliance having a coated dental composite thereon. Additional examples of assemblies (e.g., appliances) and sets of assemblies are described in U.S. Patent Application Publication No. 2005/0133384 (Cinader et al.) and U.S. Pat. No. 7,910,632 (Cinader, et al.). Packaged assemblies (e.g., orthodontic appliances) are described, for example, in U.S. Patent Application Publication No. 2003/0196914 (Tzou et al.) and U.S. Pat. Nos. 4,978,007 (Jacobs et al.), 5,015,180 (Randklev), 5,328,363 (Chester et al.), and 6,183,249 (Brennan et al.).

### Hardenable Dental Compositions

Hardenable dental compositions, or resins, are optionally present in the provided compositions, assemblies, and kits. Typically, these compositions include one or more hardenable components and a hardener. Optionally, hardenable dental compositions as described herein can include, for example, an initiator system, an ethylenically unsaturated compound, and one or more fillers. Hardenable and hardened dental compositions as described herein can be used for a variety of dental and orthodontic applications that utilize a material capable of adhering (e.g., bonding) to a tooth structure. Uses for such hardenable and hardened dental compositions include, for example, uses as adhesives (e.g., dental and/or orthodontic adhesives), cements (e.g., glass ionomer cements, resin-modified glass ionomer cements, and orthodontic cements), primers (e.g., orthodontic primers), restoratives, liners, sealants (e.g., orthodontic sealants), coatings, and combinations thereof.

Hardenable dental compositions (e.g., hardenable dental compositions) as described herein typically include a hardenable (e.g., polymerizable) component, thereby forming hardenable (e.g., polymerizable) compositions. The hardenable component can include a wide variety of chemistries, such as ethylenically unsaturated compounds (with or without acid functionality), epoxy (oxirane) resins, vinyl ethers, photopolymerization systems, redox cure systems, glass ionomer cements, polyethers, polysiloxanes, and the like. In some embodiments, the dental compositions can be hardened (e.g., polymerized by conventional photopolymerization and/or chemical polymerization techniques) prior to applying the hardened dental composition. In other embodiments, a dental composition can be hardened (e.g., polymerized by conventional photopolymerization and/or chemical polymerization techniques) after applying the hardenable dental composition.

In certain embodiments, the dental compositions are photopolymerizable, i.e., the dental compositions contain a photoinitiator (i.e., a photoinitiator system) that upon irradiation with actinic radiation initiates the polymerization (or hardening) of the dental composition. Such photopolymerizable compositions can be free radically polymerizable or cationically polymerizable. In other embodiments, the dental compositions are chemically hardenable, i.e., the dental compositions contain a chemical initiator (i.e., initiator system) that can polymerize, cure, or otherwise harden the dental composition without dependence on irradiation with actinic radiation. Such chemically hardenable dental compositions are sometimes referred to as “self-cure” compositions and may include glass ionomer cements (e.g., conventional and resin-modified glass ionomer cements), redox cure systems, and combinations thereof.

Suitable photopolymerizable components that can be used in the dental compositions as disclosed herein include, for example, epoxy resins (which contain cationically active epoxy groups), vinyl ether resins (which contain cationically active vinyl ether groups), ethylenically unsaturated compounds (which contain free radically active unsaturated groups, e.g., acrylates and methacrylates), and combinations thereof. Also suitable are polymerizable materials that contain both a cationically active functional group and a free radically active functional group in a single compound. Examples include epoxy-functional acrylates, epoxy-functional methacrylates, and combinations thereof.

Potential components of the hardenable dental composition—namely, ethylenically unsaturated compounds, ethylenically unsaturated compounds with acid functionality, epoxy (oxirane) or vinyl ether compounds, glass ionomers, photoinitiator systems, redox initiator systems, fillers, photobleachable and thermochromic dyes, and miscellaneous additives—are described further under the respective subheadings below.

### Ethylenically Unsaturated Compounds

Dental compositions as disclosed herein may include one or more hardenable components in the form of ethylenically unsaturated compounds with or without acid functionality, thereby forming hardenable dental compositions.

Suitable hardenable dental compositions may include hardenable components (e.g., photopolymerizable compounds) that include ethylenically unsaturated compounds (which contain free radically active unsaturated groups). Examples of useful ethylenically unsaturated compounds include acrylic acid esters, methacrylic acid esters, hydroxy-functional acrylic acid esters, hydroxy-functional methacrylic acid esters, and combinations thereof.

The dental compositions (e.g., photopolymerizable compositions) may include compounds having free radically active functional groups that may include monomers, oligomers, and polymers having one or more ethylenically unsaturated group. Suitable compounds contain at least one ethylenically unsaturated bond and are capable of undergoing addition polymerization. Such free radically polymerizable compounds include mono-, di- or poly-(meth)acrylates (i.e., acrylates and methacrylates) such as, methyl(meth)acrylate, ethyl acrylate, isopropyl methacrylate, n-hexyl acrylate, stearyl acrylate, allyl acrylate, glycerol triacrylate, ethyleneglycol diacrylate, diethyleneglycol diacrylate, triethyleneglycol dimethacrylate, 1,3-propanediol di(meth)acrylate, trimethylolpropane triacrylate, 1,2,4-butanetriol trimethacrylate, 1,4-cyclohexanediol diacrylate, pentaerythritol tetra(meth)acrylate, sorbitol hexaacrylate, tetrahydrofurfuryl(meth)acrylate, bis[1-(2-acryloxy)]-p-ethoxyphenyldimethylmethane, bis[1-(3-acryloxy-2-hydroxy)]-p-propoxyphenyldimethylmethane, ethoxylated bisphenol A di(meth)acrylate, and trishydroxyethyl-isocyanurate trimethacrylate; (meth)acrylamides (i.e., acrylamides and methacrylamides) such as (meth)acrylamide, methylene bis-(meth)acrylamide, and diacetone(meth)acrylamide; urethane(meth)acrylates; the bis-(meth)acrylates of polyethylene glycols (preferably of molecular weight 200-500), copolymerizable mixtures of acrylated monomers such as those in U.S. Pat. No. 4,652,274 (Boettcher et al.), acrylated oligomers such as those of U.S. Pat. No. 4,642,126 (Zador et al.), and poly(ethylenically unsaturated) carbamoyl isocyanurates such as those disclosed in U.S. Pat. No. 4,648,843 (Mitra); and vinyl compounds such as styrene, diallyl phthalate, divinyl succinate, divinyl adipate and divinyl phthalate. Other suitable free radically polymerizable compounds include siloxane-functional (meth)acrylates as disclosed, for example, in WO-00/38619 (Guggenberger et al.), WO-01/92271 (Weinmann et al.), WO-01/07444 (Guggenberger et al.), WO-00/42092 (Guggenberger et al.) and fluoropolymer-functional (meth)acrylates as disclosed, for example, in U.S. Pat. No. 5,076,844 (Fock et al.), U.S. Pat. No. 4,356,296 (Griffith et al.), EP-0373 384 (Wagenknecht et al.), EP-0201 031 (Reiners et al.), and EP-0201 778 (Reiners et al.). Mixtures of two or more free radically polymerizable compounds can be used if desired.

The hardenable component may also contain hydroxyl groups and ethylenically unsaturated groups in a single molecule. Examples of such materials include hydroxyalkyl(meth)acrylates, such as 2-hydroxyethyl(meth)acrylate and 2-hydroxypropyl(meth)acrylate; glycerol mono- or di-(meth)acrylate; trimethylolpropane mono- or di-(meth)acrylate; pentaerythritol mono-, di-, and tri-(meth)acrylate; sorbitol mono-, di-, tri-, tetra-, or penta-(meth)acrylate; and 2,2-bis[4-(2-hydroxy-3-ethacryloxypropoxy)phenyl]propane (bisGMA). Suitable ethylenically unsaturated compounds are also available from a wide variety of commercial sources, such as Sigma-Aldrich, St. Louis. Mixtures of ethylenically unsaturated compounds can be used if desired.

In certain embodiments hardenable components include PEGDMA (polyethyleneglycol dimethacrylate having a molecular weight of approximately 400), bisGMA, UDMA (urethane dimethacrylate), GDMA (glycerol dimethacrylate), TEGDMA (triethyleneglycol dimethacrylate), bisEMA6 as described in U.S. Pat. No. 6,030,606 (Holmes), and NPGDMA (neopentylglycol dimethacrylate). Various combinations of the hardenable components can be used if desired.

Preferably dental compositions as disclosed herein include at least 5% by weight, preferably at least 10% by weight, and more preferably at least 15% by weight ethylenically unsaturated compounds (e.g., with and/or without acid functionality), based on the total weight of the unfilled composition. Certain dental compositions as disclosed herein (e.g., unfilled dental compositions that consist of one or more ethylencially unsaturated compounds and an initiator system) can include 99% by weight or even higher of ethylenically unsaturated compounds (e.g., with and/or without acid functionality), based on the total weight of the unfilled composition. Other certain dental compositions as disclosed herein include at most 99% by weight, preferably at most 98% by weight, and more preferably at most 95% by weight ethylenically unsaturated compounds (e.g., with and/or without acid functionality), based on the total weight of the unfilled composition.

Ethylenically Unsaturated Compounds with Acid Functionality

Dental compositions as disclosed herein may include one or more hardenable components in the form of ethylenically unsaturated compounds with acid functionality, thereby forming hardenable dental compositions.

As used herein, ethylenically unsaturated compounds with acid functionality includes monomers, oligomers, and polymers having ethylenic unsaturation and acid and/or acid-precursor functionality. Acid-precursor functionalities include, for example, anhydrides, acid halides, and pyrophosphates. The acid functionality can include carboxylic acid functionality, phosphoric acid functionality, phosphonic acid functionality, sulfonic acid functionality, or combinations thereof.

Ethylenically unsaturated compounds with acid functionality include, for example, α,β-unsaturated acidic compounds such as glycerol phosphate mono(meth)acrylates, glycerol phosphate di(meth)acrylates, hydroxyethyl(meth)acrylate (e.g., HEMA) phosphates, bis((meth)acryloxyethyl)phosphate, ((meth)acryloxypropyl)phosphate, bis((meth)acryloxypropyl)phosphate, bis((meth)acryloxy)propyloxy phosphate, (meth)acryloxyhexyl phosphate, bis((meth)acryloxyhexyl)phosphate, (meth)acryloxyoctyl phosphate, bis((meth)acryloxyoctyl)phosphate, (meth)acryloxydecyl phosphate, bis((meth)acryloxydecyl)phosphate, caprolactone methacrylate phosphate, citric acid di- or tri-methacrylates, poly(meth)acrylated oligomaleic acid, poly(meth)acrylated polymaleic acid, poly(meth)acrylated poly(meth)acrylic acid, poly(meth)acrylated polycarboxyl-polyphosphonic acid, poly(meth)acrylated polychlorophosphoric acid, poly(meth)acrylated polysulfonate, poly(meth)acrylated polyboric acid, and the like, may be used as components in the hardenable component system. Also monomers, oligomers, and polymers of unsaturated carbonic acids such as (meth)acrylic acids, aromatic (meth)acrylated acids (e.g., methacrylated trimellitic acids), and anhydrides thereof can be used. Certain compositions for use in preferred methods of the present invention include an ethylenically unsaturated compound with acid functionality having at least one P—OH moiety.

Certain of these compounds are obtained, for example, as reaction products between isocyanatoalkyl(meth)acrylates and carboxylic acids. Additional compounds of this type having both acid-functional and ethylenically unsaturated components are described in, for example, U.S. Pat. Nos. 4,872,936 (Engelbrecht). A wide variety of such compounds containing both the ethylenically unsaturated and acid moieties can be used. Mixtures of such compounds can be used if desired.

Additional ethylenically unsaturated compounds with acid functionality include, for example, polymerizable bisphosphonic acids as disclosed for example, in U.S. Patent Application Publication No. 2004/0206932 (Abuelyaman et al.); AA:ITA:IEM (copolymer of acrylic acid:itaconic acid with pendent methacrylate made by reacting AA:ITA copolymer with sufficient 2-isocyanatoethyl methacrylate to convert a portion of the acid groups of the copolymer to pendent methacrylate groups as described, for example, in U.S. Pat. No. 5,130,347 (Mitra)); and those recited in U.S. Pat. Nos. 4,259,075 (Yamauchi et al.), 4,499,251 (Omura et al.), 4,537,940 (Omura et al.), 4,539,382 (Omura et al.), 5,530,038 (Yamamoto et al.), 6,458,868 (Okada et al.), and European Patent Application Publication Nos. EP 712,622 (Tokuyama Corp.) and EP 1,051,961 (Kuraray Co., Ltd.).

Dental compositions as disclosed herein can also include compositions that include combinations of ethylenically unsaturated compounds with acid functionality. Preferably the dental compositions are self-adhesive and are non-aqueous. For example, such compositions can include: a first compound including at least one (meth)acryloxy group and at least one —O—P(O)(OH)ₓ group, wherein x=1 or 2, and wherein the at least one —O—P(O)(OH)ₓ group and the at least one (meth)acryloxy group are linked together by a C1-C4 hydrocarbon group; a second compound including at least one (meth)acryloxy group and at least one —O—P(O)(OH)ₓ group, wherein x=1 or 2, and wherein the at least one —O—P(O)(OH)ₓ group and the at least one (meth)acryloxy group are linked together by a C5-C12 hydrocarbon group; an ethylenically unsaturated compound without acid functionality; an initiator system; and a filler. Such compositions are described, for example, in Published U.S. Application No. 2007/0248927 (Luchterhandt et al.).

Preferably dental compositions as disclosed herein include at least 5% by weight, preferably at least 10% by weight, and more preferably at least 15% by weight ethylenically unsaturated compounds with acid functionality, based on the total weight of the unfilled composition. Certain dental compositions as disclosed herein (e.g., unfilled dental compositions that consist of one or more ethylenically unsaturated compounds and an initiator system) can include 99% by weight or even higher of ethylenically unsaturated compounds with acid functionality, based on the total weight of the unfilled composition. Other certain dental compositions as disclosed herein include at most 99% by weight, preferably at most 98% by weight, and more preferably at most 95% by weight ethylenically unsaturated compounds with acid functionality, based on the total weight of the unfilled composition.

### Epoxy (Oxirane) or Vinyl Ether Compounds

Hardenable dental compositions as disclosed herein may include one or more hardenable components in the form of epoxy (oxirane) compounds (which contain cationically active epoxy groups) or vinyl ether compounds (which contain cationically active vinyl ether groups), thereby forming hardenable dental compositions.

The epoxy or vinyl ether monomers can be used alone as the hardenable component in a dental composition or in combination with other monomer classes, e.g., ethylenically unsaturated compounds as described herein, and can include as part of their chemical structures aromatic groups, aliphatic groups, cycloaliphatic groups, and combinations thereof.

Examples of epoxy (oxirane) compounds include organic compounds having an oxirane ring that is polymerizable by ring opening. These materials include monomeric epoxy compounds and epoxides of the polymeric type and can be aliphatic, cycloaliphatic, aromatic or heterocyclic. These compounds generally have, on the average, at least 1 polymerizable epoxy group per molecule, in some embodiments at least 1.5, and in other embodiments at least 2 polymerizable epoxy groups per molecule. The polymeric epoxides include linear polymers having terminal epoxy groups (e.g., a diglycidyl ether of a polyoxyalkylene glycol), polymers having skeletal oxirane units (e.g., polybutadiene polyepoxide), and polymers having pendent epoxy groups (e.g., a glycidyl methacrylate polymer or copolymer). The epoxides may be pure compounds or may be mixtures of compounds containing one, two, or more epoxy groups per molecule. The “average” number of epoxy groups per molecule is determined by dividing the total number of epoxy groups in the epoxy-containing material by the total number of epoxy-containing molecules present.

These epoxy-containing materials may vary from low molecular weight monomeric materials to high molecular weight polymers and may vary greatly in the nature of their backbone and substituent groups. Illustrative of permissible substituent groups include halogens, ester groups, ethers, sulfonate groups, siloxane groups, carbosilane groups, nitro groups, phosphate groups, and the like. The molecular weight of the epoxy-containing materials may vary from 50 to 100,000 or more.

Suitable epoxy-containing materials useful as the resin system reactive components for use in methods of the present invention are listed in U.S. Pat. Nos. 6,187,836 (Oxman et al.) and 6,084,004 (Weinmann et al.).

Other suitable epoxy resins useful as the resin system reactive components include those which contain cyclohexene oxide groups such as epoxycyclohexanecarboxylates, typified by 3,4-epoxycyclohexylmethyl-3,4-epoxycyclohexanecarboxylate, 3,4-epoxy-2-methylcyclohexylmethyl-3,4-epoxy-2-methylcyclohexane carboxylate, and bis(3,4-epoxy-6-methylcyclohexyl-methyl)adipate. For a more detailed list of useful epoxides of this nature, reference is made to U.S. Pat. Nos. 6,245,828 (Weinmann et al.), 5,037,861 (Crivello et al), and 6,779,656 (Klettke et al.).

Other epoxy resins that may be useful in dental compositions as disclosed herein include glycidyl ether monomers. Examples are glycidyl ethers of polyhydric phenols obtained by reacting a polyhydric phenol with an excess of chlorohydrin such as epichlorohydrin (e.g., the diglycidyl ether of 2,2-bis-(2,3-epoxypropoxyphenol)propane). Further examples of epoxides of this type are described in U.S. Pat. No. 3,018,262 (Schroeder), and in “Handbook of Epoxy Resins” by Lee and Neville, McGraw-Hill Book Co., New York (1967).

Other suitable epoxides useful as the resin system reactive components are those that contain silicon, useful examples of which are described in WO 01/51540 (Klettke et al.).

Additional suitable epoxides useful as the resin system reactive components include octadecylene oxide, epichlorohydrin, styrene oxide, vinyl cyclohexene oxide, glycidol, glycidylmethacrylate, diglycidyl ether of Bisphenol A and other commercially available epoxides, as provided in U.S. Pat. No. 7,262,228 (Oxman et al.).

Blends of various epoxy-containing materials are also contemplated. Examples of such blends include two or more weight average molecular weight distributions of epoxy-containing compounds, such as low molecular weight (below 200), intermediate molecular weight (200 to 10,000) and higher molecular weight (above 10,000). Alternatively or additionally, the epoxy resin may contain a blend of epoxy-containing materials having different chemical natures, such as aliphatic and aromatic, or functionalities, such as polar and non-polar.

Other types of useful hardenable components having cationically active functional groups include vinyl ethers, oxetanes, spiro-orthocarbonates, spiro-orthoesters, and the like.

If desired, both cationically active and free radically active functional groups may be contained in a single molecule. Such molecules may be obtained, for example, by reacting a di- or poly-epoxide with one or more equivalents of an ethylenically unsaturated carboxylic acid. An example of such a material is the reaction product of UVR-6105 (available from Union Carbide) with one equivalent of methacrylic acid. Commercially available materials having epoxy and free-radically active functionalities include the CYCLOMER series, such as CYCLOMER M-100, M-101, or A-200 available from Daicel Chemical, Japan, and EBECRYL-3605 available from Radcure Specialties, UCB Chemicals, Atlanta, Ga.

The cationically curable components may further include a hydroxyl-containing organic material. Suitable hydroxyl-containing materials may be any organic material having hydroxyl functionality of at least 1, and preferably at least 2. Preferably, the hydroxyl-containing material contains two or more primary or secondary aliphatic hydroxyl groups (i.e., the hydroxyl group is bonded directly to a non-aromatic carbon atom). The hydroxyl groups can be terminally situated, or they can be pendent from a polymer or copolymer. The molecular weight of the hydroxyl-containing organic material can vary from very low (e.g., 32) to very high (e.g., one million or more). Suitable hydroxyl-containing materials can have low molecular weights (i.e., from 32 to 200), intermediate molecular weights (i.e., from 200 to 10,000, or high molecular weights (i.e., above 10,000). As used herein, all molecular weights are weight average molecular weights.

The hydroxyl-containing materials may be non-aromatic in nature or may contain aromatic functionality. The hydroxyl-containing material may optionally contain heteroatoms in the backbone of the molecule, such as nitrogen, oxygen, sulfur, and the like. The hydroxyl-containing material may, for example, be selected from naturally occurring or synthetically prepared cellulosic materials. The hydroxyl-containing material should be substantially free of groups which may be thermally or photolytically unstable; that is, the material should not decompose or liberate volatile components at temperatures below 100° C. or in the presence of actinic light which may be encountered during the desired photopolymerization conditions for the polymerizable compositions.

Suitable hydroxyl-containing materials useful in methods of the present invention are listed in U.S. Pat. No. 6,187,836 (Oxman et al.).

The hardenable component(s) may also contain hydroxyl groups and cationically active functional groups in a single molecule. An example is a single molecule that includes both hydroxyl groups and epoxy groups.

### Glass Ionomers

Hardenable dental compositions as described herein may include glass ionomer cements such as conventional glass ionomer cements that typically employ as their main ingredients a homopolymer or copolymer of an ethylenically unsaturated carboxylic acid (e.g., poly acrylic acid, copoly (acrylic, itaconic acid), and the like), a fluoroaluminosilicate (“FAS”) glass, water, and a chelating agent such as tartaric acid. Conventional glass ionomers (i.e., glass ionomer cements) typically are supplied in powder/liquid formulations that are mixed just before use.

The glass ionomer cements may also include resin-modified glass ionomer (“RMGI”) cements. Like a conventional glass ionomer, a RMGI cement employs an FAS glass. However, the organic portion of an RMGI is different. In one type of RMGI, the polycarboxylic acid is modified to replace or end-cap some of the acidic repeating units with pendent curable groups and a photoinitiator is added to provide a second cure mechanism. Acrylate or methacrylate groups are usually employed as the pendant curable group. In another type of RMGI, the cement includes a polycarboxylic acid, an acrylate or methacrylate-functional monomer and a photoinitiator, e.g., as in Mathis et al., “Properties of a New Glass Ionomer/Composite Resin Hybrid Restorative”, Abstract No. 51, J. Dent Res., 66:113 (1987) and as in U.S. Pat. Nos. 5,063,257 (Akahane et al.), 5,520,725 (Kato et al.), 5,859,089 (Qian), 5,925,715 (Mitra), and 5,962,550 (Akahane et al.). In another type of RMGI, the cement may include a polycarboxylic acid, an acrylate or methacrylate-functional monomer, and a redox or other chemical cure system, e.g., as described in U.S. Pat. Nos. 5,154,762 (Mitra et al.), 5,520,725 (Kato et al.), and 5,871,360 (Kato). In another type of RMGI, the cement may include various monomer-containing or resin-containing components as described in U.S. Pat. Nos. 4,872,936 (Engelbrecht), 5,227,413 (Mitra), 5,367,002 (Huang et al.), and 5,965,632 (Orlowski). Dental compositions including such cements are able to harden in the dark due to the ionic reaction between the acidic repeating units of the polycarboxylic acid and cations leached from the glass, and commercial RMGI products typically also cure on exposure of the cement to light from a dental curing lamp. RMGI cements that contain a redox cure system and that can be cured in the dark without the use of actinic radiation are described in U.S. Pat. No. 6,765,038 (Mitra).

In certain embodiments, RMGI cements are formulated as powder/liquid or paste/paste systems, and contain water as mixed and applied. For embodiments in which the assembly includes a compressible material having the hardenable material applied thereto, water may be separated from the resin and filler. In other certain embodiments, cements having good shelf stability can be prepared by suspending water in the resin using an emulsifier to create a water-in-oil microemulsion. For other embodiments, in which the hardenable material contains no water, excess water present on the teeth can provide water for the bonding process. Fluoroaluminosilicate glass may be incorporated as an additional particulate filler or as a fibrous compressible material.

### Photoinitiator Systems

In certain embodiments, the dental compositions of the present invention are photopolymerizable, i.e., the dental compositions contain a photopolymerizable component and a photoinitiator (i.e., a photoinitiator system) that upon irradiation with actinic radiation initiates the polymerization (or hardening) of the dental composition. Such photopolymerizable compositions can be free radically polymerizable or cationically polymerizable.

Suitable photoinitiators (i.e., photoinitiator systems that include one or more compounds) for polymerizing free radically photopolymerizable compositions include binary and tertiary systems. Typical tertiary photoinitiators include an iodonium salt, a photosensitizer, and an electron donor compound as described in U.S. Pat. No. 5,545,676 (Palazzotto et al.). Preferred iodonium salts are the diaryl iodonium salts, e.g., diphenyliodonium chloride, diphenyliodonium hexafluorophosphate, diphenyliodonium tetrafluoroborate, and tolylcumyliodonium tetrakis(pentafluorophenyl)borate. Preferred photosensitizers are monoketones and diketones that absorb some light within a range of 400 nm to 520 nm (preferably, 450 nm to 500 nm). More preferred compounds are alpha diketones that have some light absorption within a range of 400 nm to 520 nm (even more preferably, 450 to 500 nm). Preferred compounds are camphorquinone, benzil, furil, 3,3,6,6-tetramethylcyclohexanedione, phenanthraquinone, 1-phenyl-1,2-propanedione and other 1-aryl-2-alkyl-1,2-ethanediones, and cyclic alpha diketones. Preferred electron donor compounds include substituted amines, e.g., ethyl dimethylaminobenzoate. Other suitable tertiary photoinitiator systems useful for photopolymerizing cationically polymerizable resins are described, for example, in U.S. Pat. No. 6,765,036 (Dede et al.).

Other suitable photoinitiators for polymerizing free radically photopolymerizable compositions include the class of phosphine oxides that typically have a functional wavelength range of 380 nm to 1200 nm. Preferred phosphine oxide free radical initiators with a functional wavelength range of 380 nm to 450 nm are acyl and bisacyl phosphine oxides such as those described in U.S. Pat. Nos. 4,298,738 (Lechtken et al.), 4,324,744 (Lechtken et al.), 4,385,109 (Lechtken et al.), 4,710,523 (Lechtken et al.), 4,737,593 (Ellrich et al.), and 6,251,963 (Kohler et al.); and EP No. 0 173 567 (Ying).

Commercially available phosphine oxide photoinitiators capable of free-radical initiation when irradiated at wavelength ranges of greater than 380 nm to 450 nm include bis(2,4,6-trimethylbenzoyl)phenyl phosphine oxide (IRGACURE 819, Ciba Specialty Chemicals, Tarrytown, N.Y.), bis(2,6-dimethoxybenzoyl)-(2,4,4-trimethylpentyl)phosphine oxide (CGI 403, Ciba Specialty Chemicals), a 25:75 mixture, by weight, of bis(2,6-dimethoxybenzoyl)-2,4,4-trimethylpentyl phosphine oxide and 2-hydroxy-2-methyl-1-phenylpropan-1-one (IRGACURE 1700, Ciba Specialty Chemicals), a 1:1 mixture, by weight, of bis(2,4,6-trimethylbenzoyl)phenyl phosphine oxide and 2-hydroxy-2-methyl-1-phenylpropane-1-one (DAROCUR 4265, Ciba Specialty Chemicals), and ethyl 2,4,6-trimethylbenzylphenyl phosphinate (LUCIRIN LR8893X, BASF Corp., Charlotte, N.C.).

Typically, the phosphine oxide initiator is present in the photopolymerizable composition in catalytically effective amounts, such as from 0.1 weight percent to 5.0 weight percent, based on the total weight of the dental composition.

Tertiary amine reducing agents may be used in combination with an acylphosphine oxide. Illustrative tertiary amines useful in the invention include ethyl 4-(N,N-dimethylamino)benzoate and N,N-dimethylaminoethyl methacrylate. When present, the amine reducing agent is present in the photopolymerizable composition in an amount from 0.1 weight percent to 5.0 weight percent, based on the total weight of the dental composition. Useful amounts of other initiators are well known to those of skill in the art.

Suitable photoinitiators for polymerizing cationically photopolymerizable compositions include binary and tertiary systems. Typical tertiary photoinitiators include an iodonium salt, a photosensitizer, and an electron donor compound as described in EP 0 897 710 (Weinmann et al.); in U.S. Pat. Nos. 5,856,373 (Kaisaki et al.), 6,084,004 (Weinmann et al.), 6,187,833 (Oxman et al.), 6,187,836 (Oxman et al.); and 6,765,036 (Dede et al.). The dental compositions of the invention can include one or more anthracene-based compounds as electron donors. In some embodiments, the dental compositions comprise multiple-substituted anthracene compounds or a combination of a substituted anthracene compound with unsubstituted anthracene. The combination of these mixed-anthracene electron donors as part of a photoinitiator system provides significantly enhanced cure depth and cure speed and temperature insensitivity when compared to comparable single-donor photoinitiator systems in the same matrix. Such compositions with anthracene-based electron donors are described in U.S. Pat. No. 7,262,228 (Oxman et al.).

Suitable iodonium salts include tolylcumyliodonium tetrakis(pentafluorophenyl)borate, tolylcumyliodonium tetrakis(3,5-bis(trifluoromethyl)-phenyl)borate, and the diaryl iodonium salts, e.g., diphenyliodonium chloride, diphenyliodonium hexafluorophosphate, diphenyliodonium hexafluoroantimonate, and diphenyliodonium tetrafluoroborate. Suitable photosensitizers are monoketones and diketones that absorb some light within a range of 450 nm to 520 nm (preferably, 450 nm to 500 nm). More suitable compounds are alpha diketones that have some light absorption within a range of 450 nm to 520 nm (even more preferably, 450 nm to 500 nm). Preferred compounds are camphorquinone, benzil, furil, 3,3,6,6-tetramethylcyclohexanedione, phenanthraquinone and other cyclic alpha diketones. Most preferred is camphorquinone. Suitable electron donor compounds include substituted amines, e.g., ethyl 4-(dimethylamino)benzoate and 2-butoxyethyl 4-(dimethylamino)benzoate; and polycondensed aromatic compounds (e.g. anthracene).

The initiator system is present in an amount sufficient to provide the desired rate of hardening (e.g., polymerizing and/or crosslinking). For a photoinitiator, this amount will be dependent in part on the light source, the thickness of the layer to be exposed to radiant energy, and the extinction coefficient of the photoinitiator. Preferably, the initiator system is present in a total amount of at least 0.01 wt-%, more preferably, at least 0.03 wt-%, and most preferably, at least 0.05 wt-%, based on the weight of the dental composition. Preferably, the initiator system is present in a total amount of no more than 10 wt-%, more preferably, no more than 5 wt-%, and most preferably, no more than 2.5 wt-%, based on the weight of the dental composition.

### Redox Initiator Systems

In certain embodiments, the dental compositions of the present invention are chemically hardenable, i.e., the dental compositions contain a chemically hardenable component and a chemical initiator (i.e., initiator system) that can polymerize, cure, or otherwise harden the dental composition without dependence on irradiation with actinic radiation. Such chemically hardenable dental compositions are sometimes referred to as “self-cure” compositions and may include glass ionomer cements, resin-modified glass ionomer cements, redox cure systems, and combinations thereof.

The chemically hardenable dental compositions may include redox cure systems that include a hardenable component (e.g., an ethylenically unsaturated polymerizable component) and redox agents that include an oxidizing agent and a reducing agent. Suitable hardenable components, redox agents, optional acid-functional components, and optional fillers are described in U.S. Pat. Nos. 7,173,074 (Mitra et al.) and 6,982,288 (Mitra et al.).

The reducing and oxidizing agents should react with or otherwise cooperate with one another to produce free-radicals capable of initiating polymerization of the resin system (e.g., the ethylenically unsaturated component). This type of cure is a dark reaction, that is, it is not dependent on the presence of light and can proceed in the absence of light. The reducing and oxidizing agents are preferably sufficiently shelf-stable and free of undesirable colorization to permit their storage and use under typical dental conditions. They should be sufficiently miscible with the resin system to permit ready dissolution in (and discourage separation from) the other components of the hardenable dental composition.

Useful reducing agents include ascorbic acid, ascorbic acid derivatives, and metal complexed ascorbic acid compounds as described in U.S. Pat. No. 5,501,727 (Wang et al.); amines, especially tertiary amines, such as 4-tert-butyl dimethylaniline; aromatic sulfinic salts, such as p-toluenesulfinic salts and benzenesulfinic salts; thioureas, such as 1-ethyl-2-thiourea, tetraethyl thiourea, tetramethyl thiourea, 1,1-dibutyl thiourea, and 1,3-dibutyl thiourea; and mixtures thereof. Other secondary reducing agents may include cobalt (II) chloride, ferrous chloride, ferrous sulfate, hydrazine, hydroxylamine (depending on the choice of oxidizing agent), salts of a dithionite or sulfite anion, and mixtures thereof. Preferably, the reducing agent is an amine.

Suitable oxidizing agents will also be familiar to those skilled in the art, and include but are not limited to persulfuric acid and salts thereof, such as sodium, potassium, ammonium, cesium, and alkyl ammonium salts. Additional oxidizing agents include peroxides such as benzoyl peroxides, hydroperoxides such as cumyl hydroperoxide, t-butyl hydroperoxide, and amyl hydroperoxide, as well as salts of transition metals such as cobalt (III) chloride and ferric chloride, cerium (IV) sulfate, perboric acid and salts thereof, permanganic acid and salts thereof, perphosphoric acid and salts thereof, and mixtures thereof.

It may be desirable to use more than one oxidizing agent or more than one reducing agent. Small quantities of transition metal compounds may also be added to accelerate the rate of redox cure. In some embodiments it may be preferred to include a secondary ionic salt to enhance the stability of the polymerizable composition as described in U.S. Pat. No. 6,982,288 (Mitra et al.).

The reducing and oxidizing agents are present in amounts sufficient to permit an adequate free-radical reaction rate. This can be evaluated by combining all of the ingredients of the hardenable dental composition except for the optional filler, and observing whether or not a hardened mass is obtained.

Preferably, the reducing agent is present in an amount of at least 0.01% by weight, and more preferably at least 0.1% by weight, based on the total weight (including water) of the components of the hardenable dental composition. Preferably, the reducing agent is present in an amount of no greater than 10% by weight, and more preferably no greater than 5% by weight, based on the total weight (including water) of the components of the hardenable dental composition.

Preferably, the oxidizing agent is present in an amount of at least 0.01% by weight, and more preferably at least 0.10% by weight, based on the total weight (including water) of the components of the hardenable dental composition. Preferably, the oxidizing agent is present in an amount of no greater than 10% by weight, and more preferably no greater than 5% by weight, based on the total weight (including water) of the components of the hardenable dental composition.

The reducing or oxidizing agents can be microencapsulated as described in U.S. Pat. No. 5,154,762 (Mitra et al.). This will generally enhance shelf stability of the hardenable dental composition, and if necessary permit packaging the reducing and oxidizing agents together. For example, through appropriate selection of an encapsulant, the oxidizing and reducing agents can be combined with an acid-functional component and optional filler and kept in a storage-stable state. Likewise, through appropriate selection of a water-insoluble encapsulant, the reducing and oxidizing agents can be combined with an FAS glass and water and maintained in a storage-stable state.

A redox cure system can be combined with other cure systems, e.g., with a hardenable dental composition such as described U.S. Pat. No. 5,154,762 (Mitra et al.).

### Fillers

In certain preferred embodiments, the hardenable dental composition is unfilled. In other certain embodiments, the hardenable dental composition further includes a filler. Fillers can be selected from one or more of a wide variety of materials suitable for incorporation in compositions used for dental applications, such as fillers currently used in dental restorative compositions, and the like.

The filler is preferably finely divided. The filler can have a unimodal or polymodal (e.g., bimodal) particle size distribution. Preferably, the maximum particle size (the largest dimension of a particle, typically, the diameter) of the filler is less than 30 micrometers, more preferably less than 20 micrometers, and most preferably less than 10 micrometers. Preferably, the average particle size of the filler is less than 0.1 micrometers, and more preferably less than 0.075 micrometer.

The filler can be an inorganic material. It can also be a crosslinked organic material that is insoluble in the resin system (i.e., the hardenable components), and is optionally filled with inorganic filler. The filler should in any event be nontoxic and suitable for use in the mouth. The filler can be radiopaque or radiolucent.

Examples of suitable inorganic fillers are naturally occurring or synthetic materials including, but not limited to: quartz (i.e., silica, SiO₂); nitrides (e.g., silicon nitride); glasses and fillers derived from, for example, Zr, Sr, Ce, Sb, Sn, Ba, Zn, and Al; feldspar; borosilicate glass; kaolin; talc; zirconia; titania; low Mohs hardness fillers such as those described in U.S. Pat. No. 4,695,251 (Randklev); and submicrometer silica particles (e.g., pyrogenic silicas such as those available under the trade designations AEROSIL, including “OX 50,” “130,” “150” and “200” silicas from Degussa Corp., Akron, Ohio and CAB-O-SIL M5 silica from Cabot Corp., Tuscola, Ill.). Examples of suitable organic filler particles include filled or unfilled pulverized polycarbonates, polyepoxides, and the like. Further examples of fillers include soft fillers as described, for example, in WO2010/039395 (Amos et al.).

Preferred non-acid-reactive filler particles are quartz (i.e., silica), submicrometer silica, zirconia, submicrometer zirconia, and non-vitreous microparticles of the type described in U.S. Pat. No. 4,503,169 (Randklev). Mixtures of these non-acid-reactive fillers are also contemplated, as well as combination fillers made from organic and inorganic materials.

The filler can also be an acid-reactive filler. Suitable acid-reactive fillers include metal oxides, glasses, and metal salts. Typical metal oxides include barium oxide, calcium oxide, magnesium oxide, and zinc oxide. Typical glasses include borate glasses, phosphate glasses, and fluoroaluminosilicate (“FAS”) glasses. FAS glasses are particularly preferred. The FAS glass typically contains sufficient elutable cations so that a hardened dental composition will form when the glass is mixed with the components of the hardenable dental composition. The glass also typically contains sufficient elutable fluoride ions so that the hardened dental composition may provide cariostatic properties. The glass can be made from a melt containing fluoride, alumina, and other glass-forming ingredients using techniques familiar to those skilled in the FAS glassmaking art. The FAS glass typically is in the form of particles that are sufficiently finely divided so that they can conveniently be mixed with the other cement components and will perform well when the resulting mixture is used in the mouth.

Generally, the average particle size (typically, diameter) for the FAS glass is no greater than 12 micrometers, typically no greater than 10 micrometers, and more typically no greater than 5 micrometers as measured using, for example, a sedimentation analyzer. Suitable FAS glasses will be familiar to those skilled in the art, and are available from a wide variety of commercial sources, and many are found in currently available glass ionomer cements such as those commercially available as VITREMER, VITREBOND, RELY X LUTING CEMENT, RELY X LUTING PLUS CEMENT, PHOTAC-FIL QUICK, KETAC-MOLAR, and KETAC-FIL PLUS (3M ESPE, St. Paul, Minn.), FUJI II LC and FUJI IX (G-C Dental Industrial Corp., Tokyo, Japan) and CHEMFIL Superior (Dentsply International, York, Pa.). Mixtures of fillers can be used if desired.

The surface of the filler particles can also be treated with a coupling agent to enhance the bond between the filler and the resin. The use of suitable coupling agents include gamma-methacryloxypropyltrimethoxysilane, gamma-mercaptopropyltriethoxysilane, gamma-aminopropyltrimethoxysilane, and the like. Silane-treated zirconia-silica (ZrO₂—SiO₂) filler, silane-treated silica filler, silane-treated zirconia filler, and combinations thereof are especially preferred in certain embodiments.

Other suitable fillers are disclosed in U.S. Pat. Nos. 6,387,981 (Zhang et al.) and 6,572,693 (Wu et al.) as well as WO 01/30305 (Zhang et al.), WO 01/30306 (Windisch et al.), WO 01/30307 (Zhang et al.), and WO 03/063804 (Wu et al.). Filler components described in these references include nanosized silica particles, nanosized metal oxide particles, and combinations thereof. Suitable nanofillers are also described in U.S. Pat. Nos. 7,090,721 (Craig et al.), 7,090,722 (Budd et al.), and 7,156,911 (Kangas et al.), as well as U.S. Published Application No. 2005/0256223 (Kolb et al.).

For embodiments in which the hardenable dental composition includes one or more fillers, the hardenable dental composition preferably includes at least 1% by weight filler, more preferably at least 2% by weight filler, and most preferably at least 5% by weight filler. For embodiments in which the hardenable dental composition includes one or more fillers, the hardenable dental composition preferably includes at most 85% by weight filler, more preferably at most 50% by weight filler, and most preferably at most 25% by weight filler.

In certain preferred embodiments, unfilled or lightly filled hardenable dental compositions provide for easy cleanup of excess hardenable and/or hardened dental composition. Lightly filled hardenable dental compositions include at most 35% by weight filler, more preferably at most 20% by weight filler, and most preferably at most 10% by weight filler. Examples of unfilled and/or lightly filled hardenable dental compositions include primers and/or self-etching primers.

In certain preferred embodiments, the hardenable dental composition (e.g., filled or unfilled) is flowable during application, for example, at oral temperatures (e.g., 37° C.) in the methods described herein. As used herein, a “flowable” hardenable dental composition means that the dental composition deforms or flows under its own weight at oral temperatures (e.g., 37° C.). Certain “flowable” hardenable dental compositions deform or flow under their own weight at room temperature (e.g., 20-25° C.).

### Photobleachable and Thermochromic Dyes

In some embodiments, hardenable dental compositions of the present invention preferably have an initial color remarkably different than dental structures. Color is preferably imparted to the dental composition through the use of an effective amount of a photobleachable or thermochromic dye. The dental composition preferably includes at least 0.001% by weight photobleachable or thermochromic dye, and more preferably at least 0.002% by weight photobleachable or thermochromic dye, based on the total weight of the dental composition. The dental composition preferably includes at most 1% by weight photobleachable or thermochromic dye, and more preferably at most 0.1% by weight photobleachable or thermochromic dye, based on the total weight of the dental composition. The amount of photobleachable and/or thermochromic dye may vary depending on its extinction coefficient, the ability of the human eye to discern the initial color, and the desired color change. Suitable thermochromic dyes are disclosed, for example, in U.S. Pat. No. 6,670,436 (Burgath et al.).

For embodiments including a photobleachable dye, the color formation and bleaching characteristics of the photobleachable dye varies depending on a variety of factors including, for example, acid strength, dielectric constant, polarity, amount of oxygen, and moisture content in the atmosphere. However, the bleaching properties of the dye can be readily determined by irradiating the dental composition and evaluating the change in color. Preferably, at least one photobleachable dye is at least partially soluble in a hardenable resin.

Exemplary photobleachable dyes are disclosed, for example, in U.S. Pat. Nos. 6,331,080 (Cole et al.), 6,444,725 (Trom et al.), and 6,528,555 (Nikutowski et al.). Preferred dyes include, for example, Rose Bengal, Methylene Violet, Methylene Blue, Fluorescein, Eosin Yellow, Eosin Y, Ethyl Eosin, Eosin bluish, Eosin B, Erythrosin B, Erythrosin Yellowish Blend, Toluidine Blue, 4′,5′-Dibromofluorescein, and combinations thereof.

Preferably, the dental composition's color change is initiated using actinic radiation using, for example, a dental curing light which emits visible or near infrared (IR) light for a sufficient amount of time. The mechanism that initiates the color change in the dental compositions of the invention may be separate from or substantially simultaneous with the hardening mechanism that hardens the resin. For example, a composition may harden when polymerization is initiated chemically (e.g., redox initiation) or thermally, and the color change from an initial color to a final color may occur subsequent to the hardening process upon exposure to actinic radiation.

The change in composition color from an initial color to a final color is preferably quantified by a color test. Using a color test, a value of ΔE* is determined, which indicates the total color change in a 3-dimensional color space. The human eye can detect a color change of approximately 3 ΔE* units in normal lighting conditions. The dental compositions of the present invention are preferably capable of having a color change, ΔE*, of at least 20; more preferably, ΔE* is at least 30; most preferably ΔE* is at least 40.

### Miscellaneous Additives

Optionally, compositions of the present invention may contain one or more of solvents (e.g., alcohols (e.g., propanol, ethanol), ketones (e.g., acetone, methyl ethyl ketone), esters (e.g., ethyl acetate), other nonaqueous solvents (e.g., dimethylformamide, dimethylacetamide, dimethylsulfoxide, 1-methyl-2-pyrrolidinone)), and water.

If desired, the dental compositions of the invention can contain additives such as indicators, dyes, pigments, inhibitors, accelerators, viscosity modifiers, wetting agents, buffering agents, stabilizers, and other similar ingredients that will be apparent to those skilled in the art. Viscosity modifiers include the thermally responsive viscosity modifiers (such as PLURONIC F-127 and F-108 available from BASF Wyandotte Corp., Parsippany, N.J.) and may optionally include a polymerizable moiety on the modifier or a polymerizable component different than the modifier. Such thermally responsive viscosity modifiers are described in U.S. Pat. No. 6,669,927 (Trom et al.) and U.S. Patent Application Publication No. 2004/0151691 (Oxman et al.).

Additionally, medicaments or other therapeutic substances can be optionally added to the dental compositions. Examples include, but are not limited to, fluoride sources, whitening agents, anticaries agents (e.g., xylitol), calcium sources, phosphorus sources, remineralizing agents (e.g., calcium phosphate compounds), enzymes, breath fresheners, anesthetics, clotting agents, acid neutralizers, chemotherapeutic agents, immune response modifiers, thixotropes, polyols, anti-inflammatory agents, antimicrobial agents (in addition to the antimicrobial lipid component), antifungal agents, agents for treating xerostomia, desensitizers, and the like, of the type often used in dental compositions. Combination of any of the above additives may also be employed. The selection and amount of any one such additive can be selected by one of skill in the art to accomplish the desired result without undue experimentation.

### Methods of Use

Dental articles (e.g., orthodontic appliances) having a compressible material attached to the surface thereof may be bonded to a tooth structure using methods (e.g., direct or indirect bonding methods) that are known in the art.

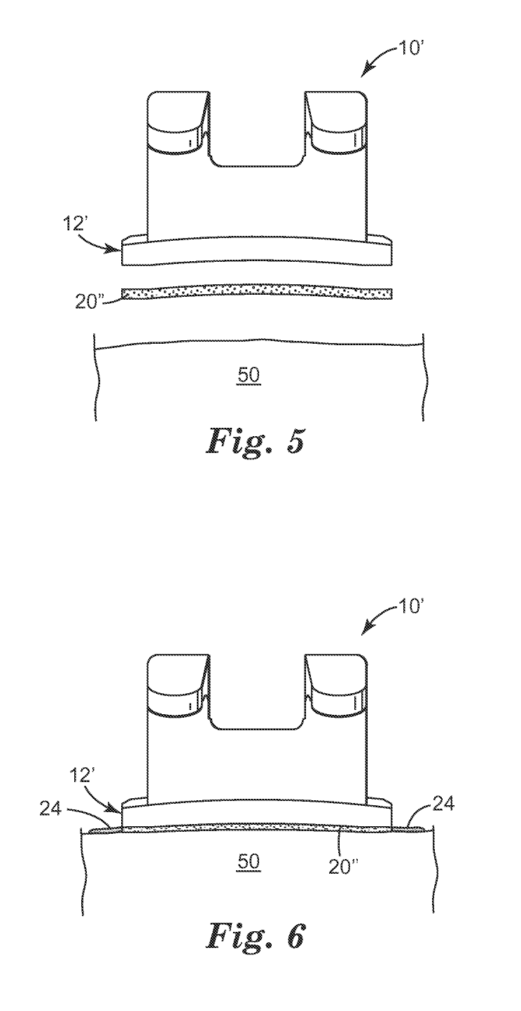

In the embodiment shown in FIG. 5, a hardenable dental composition is in contact with a coated dental composite 20″. The coated dental composite 20″ is shown in FIG. 5 prior to being attached to a base 12′ of an orthodontic appliance 10′. However, the orthodontic appliance 10′ can optionally be provided as an assembly having the dental composite 20″ pre-attached to the base 12′ of the appliance 10′ as described herein. For embodiments that encompass indirect bonding methods, the base 12′ of the appliance 10′ can be a custom base, which can also be formed from a compressed dental composite 20″ if desired.

In one embodiment, the dental composite 20″ (either alone or attached to the base 12′ of the appliance 10′) is provided having a hardenable dental composition therein. As previously indicated, however, a hardenable dental composition can be added to the compressible material of the dental composite 20″ (either alone or attached to the base 12′ of orthodontic appliance 10) by a practitioner.

A dental article (e.g., an orthodontic appliance) can be bonded to a tooth structure using compressible materials and hardenable dental compositions as described herein, using direct or indirect methods. For the embodiment illustrated in FIG. 5, the dental composite 20″ is placed in contact with a tooth structure 50 and the base 12′ of the orthodontic appliance 10′ and the hardenable dental composition is hardened. During this procedure, the orthodontic appliance 10′ is urged against the tooth structure 50 with sufficient pressure to substantially fill any gaps between the appliance 10′ and the tooth structure as illustrated in FIG. 6. Because the contour of the tooth structure surface may not precisely match the contour of the outer surface of the base 12′, the dental composite 20″ can be essentially fully compressed over some areas, and less than fully compressed in other areas.

In certain embodiments, the dental composite 20″ is compressed as completely as possible to minimize the distance between the appliance 10′ and tooth structure 50. Minimizing this distance can be advantageous to maximize bond strength and accurately express the prescription of the appliance. For certain embodiments, the dental composite 20″ can have an initial (uncompressed) thickness of 0.8 millimeters (0.03 inch) to 2.5 millimeters (0.1 inch), and a compressed thickness in at least some portions of 0.12 millimeters (0.005 inch) to 0.25 millimeters (0.01 inch) (e.g., a compressed thickness that is 0.1 times the uncompressed thickness). As shown in FIG. 6, compressing the dental composite 20″ can form one or more fillets 24 as the hardenable dental composition exudes from the dental composite 20″ onto the tooth structure 50 along the periphery of the appliance 10′.

In some embodiments, pressure is applied to the compressible material during hardening to prevent rebound of the compressible material. In other embodiments, the compressible material will remain compressed even after pressure is relieved. The use of the coated dental composite 20″ is especially advantageous in retaining excess hardenable dental composition during the process of seating the appliance 10′ on the tooth structure 50. Because the hardenable dental composition displays favorable wetting behavior with the inner and outer surfaces of the compressible material, the fillets 24 remain in contact with the periphery of the dental composite 20″ even after the appliance 10′ is fully compressed against the tooth structure 50. Consequently, the fillets 24 can act as reservoirs which allow re-absorption of expelled dental hardenable composition if and when the dental composite 20″ rebounds. Advantageously, such re-absorption can prevent voids, or air pockets, from developing along the periphery of the dental composite 20″ during the placement of the appliance 10′ on the tooth structure 50.

Preferably, the compressible material when dry displays an average rebound of preferably at most 80 percent, more preferably at most 75 percent, and most preferably at most 70 percent, when the dental composite or assembly is essentially fully compressed and subsequently allowed to relax for 60 seconds at ambient temperatures.

The tooth structure 50 can be untreated or treated. In some embodiments the tooth structure 50 is treated with a self-etching primer prior to contacting the dental composite 20″ with the tooth structure 50. In such embodiments, the hardenable dental composition can be hardened during or immediately after compressing the compressible material. In some embodiments, the hardenable dental composition is self-etching, and the tooth structure can be untreated prior to bonding the appliance 10′. For such embodiments, the hardenable dental composition preferably contacts the tooth structure for a period of time (e.g., 15 seconds or more) prior to hardening the hardenable dental composition to provide adequate time for enamel etching.

Upon application of the orthodontic appliance 10′ to the tooth structure 50, the hardenable dental composition and/or compressible material can be hardened to adhere the orthodontic appliance to the tooth structure. A variety of suitable methods of hardening the dental composition are known in the art. For example, in some embodiments the hardenable dental composition can be hardened by exposure to UV or visible light. In other embodiments, the hardenable dental composition can be provided as a multi-part composition that hardens upon combining the two or more parts.

The compressible materials as described herein can be used for indirect bonding methods. For indirect bonding methods, orthodontic appliances can be placed, for example, on a model (e.g., replica plaster or “stone” model) of the patient's dental arch to provide a custom base for later mounting on the patient's tooth structure, commonly using a placement device. In one embodiment, the orthodontic appliances have a compressible material attached to the bases thereof for bonding to the replica plaster or “stone” model. Thus, the compressible material can be compressed to form a custom base, for example, upon hardening of a hardenable dental composition. Exemplary indirect bonding methods are described, for example, in U.S. Pat. No. 7,137,812 (Cleary et al.). In another embodiment, brackets are held in place on the model during formation of the placement device using a temporary adhesive. The compressible material and hardenable composition can be added to the bracket base at any time between removal from the model and insertion in the patient's mouth.

In another embodiment, an indirect bonding placement device can be formed about a rapid prototyping model (e.g., prepared by stereolithography, selective laser sintering, fused deposition modeling, and the like, or combinations thereof) of the patient's teeth with appliances attached. Such a rapid prototyping model can be produced from data supplied by a scan of an impression of the patient's teeth, a model of the patient's teeth, or of the teeth directly. Brackets can be held in place during formation of the placement device, for example, by a temporary adhesive or by friction fit with the guides as described, for example, in Published U.S. Patent Application No. 2006/0257821 (Cinader et al.). Compressible material can be added to the bracket bases following removal from the stereolithography model. For embodiments in which the brackets are held in place by friction fit with the placement guides, compressible material can be attached to the brackets prior to placement in the guides. If not already present, a hardenable dental composition can be added to the compressible material at any time from immediately following removal from the rapid prototyping model to immediately prior to placement in the patient's mouth.

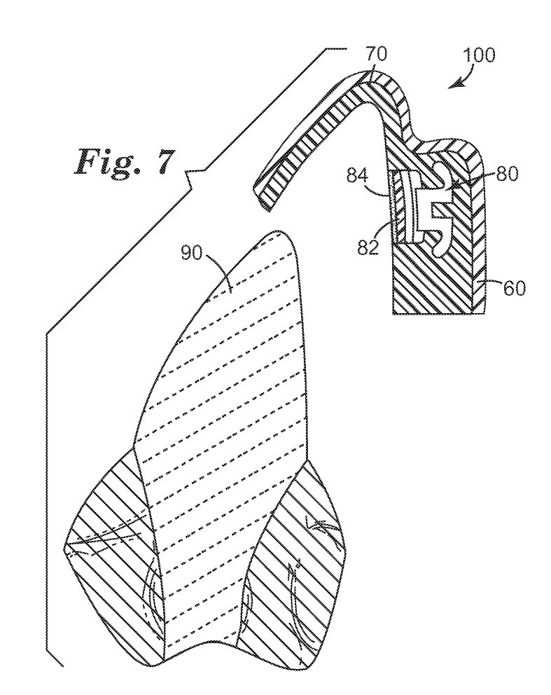

FIG. 7 shows an embodiment in which an orthodontic assembly 80 provided in a placement device 100 includes a coated dental composite 84 attached to a base such as provided by a custom lingual appliance (which can optionally be formed from a compressed, compressible material) for bonding to a patient's tooth. In FIG. 7, the placement device 100 (comprising a shell 60, matrix material 70, and one or more assemblies 80) is shown in cross-sectional view. The assemblies 80 include appliances having custom bases 82 having the dental composite 84 attached thereto. The assemblies 80 can optionally include a hardenable dental composition, which can optionally be in contact with the dental composite 84. The placement device 100 can then be placed in a package by the manufacturer and shipped to the practitioner's office.

Once the patient has returned to the office, the bonding procedure is undertaken. After any tooth preparation steps are completed, the package (if present) is opened and the placement device 100 is removed from the package. A hardenable dental composition can be placed in contact with the dental composite 84, for example, if the assembly 80 does not already include a hardenable dental composition in contact with the dental composite 84. The shell 60 is then positioned over a corresponding tooth 90 and seated, optionally with a swinging, hinge-type motion. Since the shape of the cavity of the matrix material 70 matches the shape of the underlying teeth, the assemblies 80 are simultaneously seated against the underlying teeth 90 at substantially the same locations corresponding to the previous position of the assemblies 80 on the replica. Preferably, pressure is then applied to the occlusal, labial and buccal surfaces of the shell 60 until such time as the dental composite 84 has been sufficiently compressed, and oftentimes until the hardenable dental composition and/or compressible material (e.g., for embodiments in which the compressible material is, for example, a foamed and optionally partially hardened dental composition) have been hardened. Optionally, finger pressure may be used to firmly press the assemblies 80 against the enamel surfaces of the patient's teeth 90.

Upon application of assemblies 80 to the enamel surfaces of the patient's teeth 90, the hardenable dental composition and/or compressible material can be hardened to adhere assemblies 80 to the enamel surfaces of the patient's teeth 90. As previously described, a variety of suitable methods of hardening the dental composition are known in the art. In some embodiments, and similarly to direct bonding methods, the hardenable dental composition can be hardened by exposure to UV or visible light. In other embodiments, the hardenable dental composition can be provided as a multi-part composition that hardens upon combining the two or more parts. This multi-part composition can take a form in which the two parts are mixed prior to adding to the compressible material or a form in which one part is applied to the compressible material and one part is applied to the teeth.

Once the hardenable dental composition has hardened, the shell 60 is carefully removed from the patient's dental arch. Preferably, the shell 60 is first separated from the matrix material 70, which remains in place over the dental arch along with the assemblies 80. Next, the matrix material 70 is detached from the assemblies 80. Optionally, a hand instrument such as a scaler may be used to help hold each assembly 80 against the surface of the respective tooth 90 of the patient as the matrix material 70 is peeled away from the assemblies 80. However, in instances where a relatively soft matrix material is employed or otherwise readily releases from the assemblies 80, the use of a scaler to help avoid fracturing the fresh adhesive bond is optional. As another option, the shell 60 may be separated from the matrix material 70 before the hardenable dental composition has hardened. This option is particularly useful when the hardenable dental composition includes a light-curable adhesive. Once the matrix material 70 has been detached from the assemblies 80, an archwire is placed in the slots of the assemblies 80 (e.g., appliances) and ligated in place to initiate orthodontic treatment.

The provided embodiments are also advantageous when used with other types of indirect bonding placement devices. Examples of other useful indirect bonding placement devices are described in published U.S. Patent Application Nos. 2008/0233530 (Cinader, et al.), and 2007/0287120 (Cinader et al.), and U.S. Pat. Nos. 7,452,205 (Cinader, et al.), 7,556,496 (Cinader, et al.), 7,762,815 (Cleary et al.), and 7,845,938 (Cinader, et al.).

Advantageously, for embodiments in which the hardenable dental composition is unfilled or lightly filled, the practitioner may not need to remove excess dental composition (e.g., hardened or unhardened) from the tooth structure.

If removal of excess dental composition is desired, removal of unfilled or lightly filled dental composition (e.g., hardened or unhardened) can typically be effected by rinsing with water, applying toothpaste, brushing, or a combination thereof, by the practitioner or patient, which can reduce the risk of dislodging the appliance and/or damaging enamel that can be encountered during removal of excess highly filled hardened dental composition. In another embodiment, such excess unfilled or lightly filled hardenable or hardened dental composition can remain on the tooth as, for example, a sealant that can preferably provide additional protection to the tooth structure.

### EXAMPLES

Objects and advantages of this invention are further illustrated by the following examples. While particular materials and amounts thereof are provided herein, these should not be construed to unduly limit this invention. Unless otherwise noted, all parts and percentages are on a weight basis and all molecular weights are weight average molecular weight. Also unless otherwise noted, all solvents and reagents were obtained from Aldrich Chemical Company in Milwaukee, Wis.

As used herein,

“Ammonium Hydroxide” refers to 1.0% ammonium hydroxide solution prepared by diluting 28-30% ammonium hydroxide aqueous solution from Acros Organics in Geel, BELGIUM;

“BHT” refers to butylated hydroxytoluene, from PMC Specialties, Inc. Cincinnati, Ohio;

“BisGMA” refers to Bisphenol A DiGlycidyl Ether Methacrylate produced at 3M ESPE, Irvine, Calif.;

“CPQ” refers to camphorquinone, from Aldrich Chemical Company Mikwaukee, Wis.;

“DPI” refers to Diphenyliodonium Hexafluorophosphate from Johnson Matthey (Alfa Aesar);

“EDMAB” refers to Ethyl 4-(dimethylamino)benzoate from Aldrich Chemical Co., Milwaukee, Wis.;

“GF 31” refers to 3-methoxypropyltrimethoxsilane from Wacker Chemie AG in Munchen, GERMANY;

“RelyX” refers to 3M ESPE RELY X brand ceramic primer, from 3M Company in St. Paul, Minn.;

“SIL” refers to 3M ESPE SIL brand silane primer, from 3M Company in St. Paul, Minn.;

“Trifluoroacetic acid” refers to 1% trifluoroacetic acid aqueous solution prepared by diluting trifluoroacetic acid from Aldrich Chemical Company in St. Louis, Mo.;

“TBXT Paste” refers to Transbond XT Light Cure Adhesive, from 3M Unitek in Monrovia, Calif.;

“TBXT Primer” refers to Transbond XT Orthodontic Primer, from 3M Unitek in Monrovia, Calif.;

“TBXT Etching Gel” refers to 37% phosphoric acid gel, from 3M Unitek, Monrovia, Calif.; and

“Zr/Si Cluster Filler” refers to silane-treated zirconia/silica filler, from 3M ESPE, Irvine, Calif.

### Atomic Layer Deposition Procedure

Polypropylene nonwoven mats were made using a standard meltblown fiber forming process, as described in U.S. Patent Application Publication No. 2006/0096911 (Brey et al.). The mat used in these examples was produced with a 5.9 micrometer effective fiber diameter (EFD), 37 grams per square meter basis weight, 5.4% solidity, and 762 micrometer (30 mil) process thickness.

The polypropylene nonwoven webs were subsequently coated using an ALD process, described in International Patent Publication Nos. WO2011/037831 and WO2011/037798. In this procedure, the nonwoven mat was placed into a flow-through reactor that allows reactive gases to substantially permeate throughout the nonwoven mat, thereby contacting its inner and outer surfaces. A coating of aluminum oxide was prepared by passing trimethylaluminum through the reactor, followed by ozone (approximately 18% oxygen). This two-step cycle was repeated 25 times at 60° C. to create a substantially uniform layer of 5 nanometer thickness on the nonwoven web.

ALD-treated polypropylene nonwoven fabrics were silane treated with a basic solution in water. A 1% solution of GF 31 in water was then prepared. The pH was adjusted to approximately 9.5 using Ammonium Hydroxide. The polypropylene fabrics were immersed in this solution, and then placed on a glass slide in an 80° C. oven for 1 hour.

### Resin Preparation

Resin A was compounded, using conventional methods, according to the formulation provided in Table 1 below:

TABLE 1
Formulation of Resin A used in Examples 1-4 and Comparatives B-D.

| | Amount |
|---|---|
| Component | (weight percent) |
| BisGMA | 44.1 |
| Diacryl 101 | 44.1 |
| Zr/Si Cluster Filler | 9.8 |
| EDMAB | 1.0 |
| DPI | 0.6 |
| CPQ | 0.25 |
| BHT | 0.1 |

### Bond Strength Sample Preparation Procedure

Brackets with paste adhesive were constructed and bonded as follows:
1) Obtain CLARITY brand SL upper central orthodontic brackets (P/N 007-401, 402, 501, or 502, 3M Unitek, Monrovia, Calif.) (as sent).
2) Obtain potted bovine teeth.
3) Apply TBXT etching gel to the tooth surface. Allow the etchant to sit on the teeth for 15 seconds before rinsing and drying.
4) Apply a thin coat of TBXT Primer.
5) Apply approximately 8 mg of TBXT Paste to the bracket bonding base.
6) Press the bracket onto the tooth.
7) Clean excess adhesive flash from the periphery of the bonding base.
8) Cure the adhesive with a 5 second exposure directly to the facial side of the bracket with an ORTHOLUX brand LED curing light (3M Unitek, Monrovia, Calif.).
9) Store the samples at 37° C. overnight and debond as described below.
   Brackets with exemplary coated dental composite adhesives were constructed and bonded as follows:
1) Obtain CLARITY brand SL upper central orthodontic brackets (P/N 007-401, 402, 501, or 502, 3M Unitek, Monrovia, Calif.) (as sent).
2) Obtain potted bovine teeth.
3) Use a rotary die to cut bonding base shaped pads of the non-coated or coated polypropylene nonwoven mat, prepared according to the Atomic Layer Deposition Procedure.
4) Apply Resin A to the bonding base through a 22 gauge dispensing tip (P/N 7018260, 3M Unitek, Monrovia, Calif.) via an EFD dispenser set at a 0.1 second dispense time and a pressure of 620 kilopascals (90 psi).
5) Place the nonwoven pad onto the bonding base. Press into place with a 500 micrometer diameter fiber optic lightguide and cure the adhesive dot with a 3 second exposure from the ORTHOLUX brand Luminous curing light (3M Unitek, Monrovia, Calif.).
6) Apply approximately 3.5 milligrams of Resin A to the nonwoven pad with the EFD dispenser.
7) Place the bracket in the 60° C. oven just long enough for the adhesive to soak into the nonwoven.
8) Apply TBXT etching gel to the tooth surface. Allow the etchant to sit on the teeth for 15-30 seconds before rinsing and drying.
9) Apply a thin coat of TBXT primer.
10) Press the bracket coated with the flashless adhesive onto the tooth.
11) Release pressure on the bracket and cure the adhesive with a 5 second exposure to the facial side of the bracket with an ORTHOLUX brand LED curing light (3M Unitek, Monrovia, Calif.).

### Shear Peel Bond Strength Test Procedure

After all specimens were fully bonded according to the Bond Strength Sample Preparation Procedure, they were submerged in water maintained at 37° C. for 16-24 hours. Debonding was conducted on each test specimen using a Q-TEST brand 5 Universal Test Machine (MTS, Eden Prairie, Minn.) outfitted with a 1000 newton load cell. For each debonding, the test specimen was mounted in a fixture, then a 0.51 millimeter (0.020 inch) diameter stainless steel wire fixed to a crosshead was looped beneath the occlusal tiewings of the bracket and the crosshead was translated upwards at a speed of 5.1 millimeters (0.20 inches) per minute in a direction parallel to the tooth surface until shear failure was observed. Raw force data were converted to force per unit area (in megapascals) using the known bracket base area

To maintain consistency, all samples within a series were tested in one sitting by a single operator. For each adhesive tested, the mean and standard deviation of shear bond strength were reported for a set of at least ten replicated test measurements.

### Extraction Test Procedure

Levels of extractable components were quantified by conducting a solvent extraction study on photopolymerized samples. All samples were prepared with Resin A as described above. The samples were prepared according to the following steps:
1) Cut the polypropylene nonwoven (untreated or ALD-treated) into discs of 15 millimeter diameter.
2) To silanate some of the ALD-treated nonwovens, a 1% solution of GF 31 in water was prepared. The pH was adjusted to approximately 9.5 using Ammonium Hydroxide. The polypropylene discs were immersed in this solution, and then placed on a glass slide in an 80° C. oven for 1 hour. The silane solution was freshly made on the day of use.
3) Place a thick steel mold (15 millimeters ID×1.7 millimeters thickness) onto a polyester sheet.
4) Fill the mold with Resin A.
5) Place a stack of nine polypropylene nonwoven circles onto the polyester film within the mold.
6) Place Resin A on top surface of polypropylene nonwoven circles.
7) Place a second sheet of polyester film on top and put in an oven at 60° C. for 1-2 minutes.
8) Press the assembly together with a glass microscope slide.
9) Expose the adhesive to nine 3-second exposures at nine discrete locations on the nonwoven mat (one in the center and eight equally spaced around the perimeter) using an ORTHOLUX brand Luminous curing light on top and bottom.
10) Remove the samples from the polyester sheets and molds.
11) Wipe the disc samples to remove any dust/shards generated while removing the discs from the molds.
12) Place the discs in glass vials and separate them from one another by suspending them with a stainless steel spring.
13) Fill the vials with 8 milliliters of methanol to yield 3.0 square centimeters of the test material per milliliter of extraction solvent. Close the cap seal and the vials with vinyl tape.
14) Evaluate at three sampling times: 24 h, 72 h (3 days), and 168 h (7 days). Perform the extractions at 37° C. with mild agitation for 24 h. Following the 24 h extraction period, collect the 24 h extract and replace with fresh solvent. Continue the extraction for another 48 h (corresponding to the 72 h extraction period). Collect the 48 h extract and continue the extraction by solvent exchange for 96 h (corresponding to the 168 h extraction period).
15) Evaporation of the solvent by dried nitrogen allowed measurement of the mass of material extracted. Record the percentage of the discs lost as extractant.

### Rebound Test Sample Preparation Procedure

The following procedure was used to prepare dry and wet dental composite samples containing a hardenable dental composition (in this case, TBXT Primer) for sample rebound testing:
1) Collect 0.635 centimeter (¼ inch) diameter mats from the nonwoven web samples, in down web direction, using a 0.635 centimeter (¼ inch) diameter hand-punch.
2) Collect 1.59 centimeter (⅝ inch) diameter Scotch 1022 release liners from the roll (P/N 70200548058, 3M Company, St. Paul, Minn.) using a 1.59 centimeter (⅝ inch) diameter hand-punch.
3) Tare the analytical balance (MS 204S/03, Mettler Toledo Inc., Columbus, Ohio).
4) Place a release liner on the analytical balance with non-coated side facing up and record its weight.
5) Apply a single drop of TBXT Primer onto the release liner. Remove or add resin to reach the target amount. Record the weight of liner and resin.
6) Add a mat on the resin on the liner and record the total weight (including the liner, resin, and mat).
7) Remove the sample from the balance and place a second release liner on the sample with non-coated side facing the sample.
8) Do appropriate subtractions to obtain the weight of the mat and resin individually.

### Sample Rebound Test Procedure

Test samples were prepared according to the Rebound Test Sample Preparation Procedure above. Notably, the prepared samples each display some degree of mechanical rebound (or “spring back”) after the appliance base has been fully seated against a rigid substrate, such as a tooth surface. To measure differences in the extent of rebound, samples were analyzed using a TA.XTPlus Texture Analyzer (Texture Technologies Corp., Scarsdale, N.Y.). A stainless steel cylinder of 2.54 centimeter (1 inch) diameter was used as a mechanical probe to approximate the compound curvature of a tooth structure. For each measurement, the following steps were executed:
1) Translate probe downwards (compression) at a speed of 0.051 centimeters/s (0.020 inches/s).
2) Trigger test at force of 0 grams.
3) Begin data acquisition.
4) Continue to translate probe downwards at speed of 0.00254 centimeters/s (0.001 inches/s).
5) Stop probe when force=500 g.
6) Hold force at 500 g for 5 s.
7) Translate probe upwards (extension) at speed of 0.0127 centimeters/s (0.0050 inches/s).
8) Stop probe when force=0 g.
9) Hold force at 0 g for 60 s.
10) End data acquisition.
11) Translate probe upwards at speed of 0.051 centimeters/s (0.020 inches/s).
   Probe position was recorded at three positions: i) when the probe contacts the sample at force=0 in Step 2 (“initial thickness”), ii) 5 s after full compression at 500 g force level in Step 6 above (“fully compressed”) and iii) 60 s after reaching zero force level in Step 9 above (“fully relaxed”). Rebound is measured as the difference between sample thickness when fully relaxed and that when fully compressed divided by the initial thickness.

All probe positions were adjusted to compensate for the thickness of two layers of Scotch 1022 release film (P/N 70200548058, 3M Company, St. Paul, Minn.) used in sample preparation. Each reported measurement represents the average of at least 19 replicated sample measurements.

### Examples 1-2 and Comparatives A-B

To demonstrate the bond strength of dental composites constructed of polypropylene nonwoven materials, four adhesives were evaluated according to the Shear peel Bond Strength Test Procedure:
Coated polypropylene nonwoven mat+Resin A (Example 1)
Coated and silanated polypropylene nonwoven mat+Resin A (Example 2)
Uncoated polypropylene nonwoven mat+Resin A (Comparative A)
TBXT Paste (Comparative B)
   The shear bond strength results are shown in Table 2 below:

TABLE 2
Shear peel bond strength of Examples 1-2 and Comparatives A-B

| | |
|---|---|
| | Shear peel bond |
| Example/ | strength (megapascals) |

| Comparative | Mean | Standard Deviation |
|---|---|---|
| 1 | 11.6 | 3.3 |
| 2 | 14.3 | 3.0 |
| A | 10.8 | 4.1 |
| B | 15.2 | 3.5 |

### Examples 3-4 and Comparatives C-E

Extraction studies were conducted to investigate the effect of the conformal coating on the mass of extractable components when the cured composition is subjected to methanol over extended periods of time. The following samples were cured and then evaluated according to the Extraction Test Procedure:
Coated polypropylene nonwoven mat+Resin A (Example 3)
Coated and silanated polypropylene nonwoven mat+Resin A (Example 4)
Resin A only (Comparative C)
Uncoated polypropylene nonwoven mat+Resin A (Comparative D)
TBXT Paste (Comparative E)
   The weight fraction of each disc lost through extraction is shown in Table 3 below.

TABLE 3
Disc composition effects on residue

| Example/ | % residue (by weight) | | |
|---|---|---|---|
| Comparative | 24 h | 72 h | 168 h |
| 3 | 1.58 | 2.27 | 3.04 |
| 4 | 2.13 | 2.98 | 4.04 |
| C | 2.01 | 2.68 | 3.33 |
| D | 3.02 | 4.43 | 5.96 |
| E | 0.60 | 0.78 | 0.91 |

### Examples 5-6 and Comparatives F-G

Rebound measurements were made to compare the performance of dental composites containing coated nonwoven mats with those containing uncoated nonwoven mats. These measurements were conducted using the Sample Rebound Test Procedure on both dry samples and set samples, enumerated as follows:
Coated polypropylene nonwoven mat (Example 5)
Coated polypropylene nonwoven mat+TBXT Primer (Example 6)
Uncoated polypropylene nonwoven mat (Comparative F)
Uncoated polypropylene nonwoven mat+TBXT Primer (Comparative G)
   The rebound test results are provided in Table 4 below.

TABLE 4
Sample rebound after compression

| | | Probe position (micrometers) | | | |
|---|---|---|---|---|---|
| Example/ | Hardenable | Initial | Fully | Fully | % |
| Comparative | Composition | thickness | compressed | relaxed | Rebound |
| 5 | None | 340 ± 33 | 101 ± 16.3 | 325 ± 28.7 | 65.8 |
| F | None | 447 ± 58.4 | 85.1 ± 16.8 | 406 ± 44.5 | 71.9 |
| 6 | TBXT Primer | 345 ± 27.9 | 113 ± 16.9 | 307 ± 20.3 | 56.2 |
| G | TBXT Primer | 391 ± 55.9 | 103 ± 13.1 | 320 ± 15.1 | 55.5 |

1. (canceled)

2. A dental assembly comprising:
a dental article having an outer surface for attachment to a tooth; and
an adhesive in contact with the outer surface, the adhesive comprising:
a compressible material; and
a conformal coating disposed on at least a portion of the compressible material.

3. The composite or assembly of claim 2, wherein the conformal coating is bonded to the compressible material.

4. The composite or assembly of claim 2, further comprising a hardenable composition in contact with the conformal coating.

5. The composite or assembly of claim 2, wherein the compressible material comprises meltblown nonwoven microfibers.

6. The composite or assembly of claim 5, wherein the microfibers have an average effective fiber diameter ranging from 0.1 to 20 micrometers.

7-8. (canceled)

9. The composite or assembly of claim 6, wherein the microfibers comprise polyolefin microfibers.

10. The composite or assembly of claim 2, wherein the conformal coating comprises an inorganic coating.

11. The composite or assembly of claim 10, wherein the inorganic coating comprises an aluminum oxide coating.

12. (canceled)

13. The composite or assembly of claim 12, wherein the thickness is in the range of 2 to 20 nanometers.

14-15. (canceled)

16. The composite or assembly of claim 2, wherein the compressible material when dry displays an average rebound of at most 80 percent after the dental composite or assembly is substantially fully compressed and subsequently allowed to relax for 60 seconds at ambient temperatures.

17. The composite or assembly of claim 16, wherein the average rebound is at most 75 percent.

18. (canceled)

19. A dental assembly comprising:
a dental article having an outer surface for attachment to a tooth; and
an adhesive at least partially coated on the outer surface, the adhesive comprising:
a polymeric component; and
a conformal coating disposed on at least a portion of the polymeric component.

20. The assembly of claim 19, wherein the conformal coating is bonded to the polymeric component.

21. The assembly of claim 20, wherein the adhesive further comprises a hardenable composition in contact with the polymeric component.

22. The assembly of claim 19, wherein the conformal coating comprises an inorganic coating, and wherein the inorganic coating is silanted.

23. (canceled)

24. A method of making a dental composite, comprising:
applying a conformal coating to at least a portion of a compressible material in an amount sufficient to enhance the wetting properties of the compressible material; and
placing a hardenable composition in contact with the inorganic coating.

25. The method of claim 24, wherein applying a conformal coating comprises a stepwise atomic layer deposition of the conformal coating.

26. The method of claim 25 wherein the conformal coating comprises an inorganic coating.

27. The method of claim 26, further comprising silanating the inorganic coating to enhance adhesion between the inorganic coating and the hardenable composition.

28. The method of claim 25, wherein stepwise atomic layer deposition of the conformal coating comprises iteratively transmitting two or more reactive gases through the compressible material to induce two or more self-limiting reactions on the surface of the compressible material.

29-34. (canceled)
